(19) United States
(12) Patent Application Publication
(10) Pub. No.: US 2004/0138199 A1
(43) Pub. Date: Jul. 15, 2004

(54) Benzoxazines and derivatives thereof as therapeutic agents

(76) Inventors: Rocco Dean Gogliotti, Pinckney, MI (US); Keri Lynn Muccioli, Canton, MI (US); Kimberly Suzanne Para, Ann Arbor, MI (US); Melean Visnick, Ann Arbor, MI (US)

Correspondence Address: WARNER-LAMBERT COMPANY, 2800 PLYMOUTH RD, ANN ARBOR, MI, 48105, US

(21) Appl. No.: 10/743,852

(22) Filed: Dec. 22, 2003

Related U.S. Application Data
(60) Provisional application No. 60/435,227, filed on Dec. 20, 2002.

(51) Int. Cl. A61K 31/553; A61K 31/554; A61K 31/5513

(52) U.S. Cl. 514/211.09; 514/221; 540/552; 540/569

(57) ABSTRACT

The present invention provides compounds of Formula I

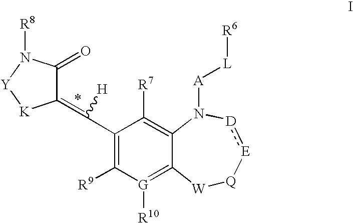

wherein W, Q, E, D, A, L, R⁶, R⁷, R⁸, Y, K, R⁹, R¹⁰, G, the dashed bond between D and E, and the double bond denoted “*” have any of the values defined therefore in the specification, and pharmaceutically acceptable salts thereof, that are useful as agents in the treatment of diseases and conditions, including inflammatory diseases, cardiovascular diseases, and cancers. Also provided are pharmaceutical compositions comprising one or more compounds of Formula I.

### CROSS-REFERENCES TO RELATED APPLICATIONS

This application claims the benefit of U.S. Provisional Patent application Serial No. 60/435,227, filed on Dec. 20, 2002, the teachings of which are herein incorporated by reference.

### BACKGROUND OF THE INVENTION

Phosphoinositide-3-kinases (PI3Ks) are a family of lipid kinases that phosphorylate phosphoinositols on the 3′-OH to generate PI3P (phosphatidylinositol 3-phosphate), PI-3,4-P2 and PI3,4,5-P3. One class of PI3Ks are stimulated by growth factors (Katso et al. Annu. Rev. Cell Dev. Biol. 2001;14:615-675) and include PI3Kα, PI3Kβ, and PI3Kδ (Vanhaesebroeck et al. Proc. Natl. Acad. Sci., U.S.A., 1997;94:4330-4335; Katso et al., 2001). A separate class of PI3Ks are activated by G-protein coupled receptors and include P13Kγ. The growth-factor stimulated PI3Ks (e.g., PI3Kα), have been implicated in cellular proliferation and cancer (reviewed in Katso et al., 2001; and Vivanco and Sawyers Nature Reviews, 2002;2:489-501). PI3Kγ has been demonstrated to be involved in signaling cascades. For example, PI3Kγ is activated in response to ligands such as C5a, fMLP, ADP, and IL-8. In addition, PI3Kγ has been implicated in immune diseases (Hirsch et al. Science 2000;287:1049-1053). PI3Kγ null macrophages show a reduced chemotactic response and a reduced ability to fight inflammation (Hirsch et al. 2000). Furthermore, PI3Kγ has also been implicated in thrombolytic diseases (e.g., thromboembolism, ischemic diseases, heart attacks, and stroke) (Hirsch et al. FASEB J. 2000;15(11):2019-2021; and Hirsch et al. FASEB J., Jul. 9, 2001;10.1096/fj.00-080fje (cited herein as Hirsch et al., 2001).

Inhibitors of members of the PI3Ks are being developed for the treatment of human disease (see e.g., WO 01/81346; WO 01/53266; and WO 01/83456). Therefore, there is a need in the art for compounds that can inhibit PI3Ks for use as pharmaceutical agents.

### SUMMARY OF THE INVENTION

In one aspect, the present invention provides for compounds of formula I:

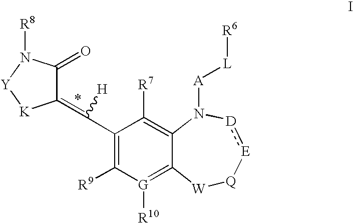

or a pharmaceutically acceptable salt thereof;

wherein W is O, S, or NR²¹;

wherein R²¹ is selected from the group consisting of: —H, —CF₃, a C₁₋₆alkyl, and phenyl;

wherein Q is (CR²R³)ₚ,

wherein R² and R³ are independently selected from H or —CH₃;

wherein p is 0 or 1;

wherein E is CR⁴R⁵;

wherein R⁴ and R⁵ are independently selected from H or —CH₃;

wherein D is CR²⁸R³⁰;

wherein R²⁸ and R³⁰ are independently selected from H or —CH₃;

wherein the dashed bond between D and E can be absent or present;

wherein A is absent, —S(O)₂—, —C(O)—, —C(O)—O—, —C(O)—NH—, or —C(S)—NH—;

wherein L is absent, a C₁-C₃-alkylene, —CH₂—, —(CH₂)₂—, —CH═CH—, a C₂-C₃-alkenylene, —CH₂—O—, —C₁-C₃-alkyl-O—, —CH₂—O—CH₂—, —C₁-C₃-alkyl-O—C₁-C₃-alkyl, —CH₂—S—, —C₁-C₃-alkyl-S—, C₁-C₃-alkyl-S(O)—, C₁-C₃-alkyl-S(O)₂—, —C₁-C₃-alkyl-S—C₁-C₃-alkyl-, —C₁-C₃alkyl-CO—,

C₁-C₃alkyl-C(O)O—, —C₁-C₃alkyl-C(O)—CH₂—, —C₁-C₃alkyl-C(O)NR²²—, —C₁-C₃alkyl-NR²²—C(O)—, —C₁-C₃alkyl-NR²²—C(O)—NR²⁴—, or —C₁-C₃alkyl-NR²²;

wherein R²² and R²⁴ are independently selected from H, and C₁₋₃alkyl;

wherein R⁶ is selected from the group consisting of H, a C₁₋₉alkyl, a C₂₋₉alkenyl, a C₂₋₉alkynyl, C(C₁-C₅alkyl)(C₁-C₅alkyl), a C₃-C₈cycloalkyl, a 3- to 8-membered heterocycloalkyl, a piperidinyl, a 6- to 11-membered bicyclic heterocycloalkyl, a 6- to 9-membered bridged bicyclic heterocycloalkyl, a 5-membered heteroaryl, a 5-isoxazole, a 3-isoxazole, an isoxazole, a 2-furanyl, a 3-furanyl, a 2-thienyl, a 3-thienyl, a thienyl, a 6-membered heteroaryl, a pyridinyl, a 4-pyridinyl, a 3-pyridinyl, an 8- to 12-membered bicyclic heteroaryl, a 2-quinoxalinyl, a quinoxalinyl, a phenyl, a naphthalenyl, a 1-naphthalenyl, a 2-naphthalenyl, a 9- to 12-membered bicyclic aryl, a 9,10-dioxo-9,10-dihydro-anthracen-2-yl, a benzofurazanyl, and a 4-(2,2-difluoro-1,3-benzodioxolyl;

wherein R⁷ is H, F, CF₃, or CH₃;

wherein R⁸ is H, —CH₂COOH, phenyl, —CH₃, a C₁₋₆alkyl, or a C₂₋₆alkenyl;

wherein Y is C(O), or C(S);

wherein K is NH, O, CH₂, or S;

wherein R⁹ is H, F, CF₃, or CH₃;

wherein G is C or N;

wherein R¹⁰ is H, —O—C₁₋₃alkyl, a C₁₋₃alkyl, —NO₂, —NR¹⁶R¹⁸, a —S—C₁₋₃alkyl, For Cl;

wherein if G is N, then R¹⁰ is absent;

wherein R¹⁶ and R¹⁸ are independently selected from the group consisting of: H, and C₁₋₃alkyl; and

wherein the stereochemistry of the double bond denoted “*” is entgegen or zusammen.

In certain embodiments, W is O, G is C, p is 0, and R⁴, R⁵, R⁷, R⁸, R⁹, R¹⁰, R²⁸, and R³⁰ are H, and the dashed bond between D and E is absent—a compound of Formula X:

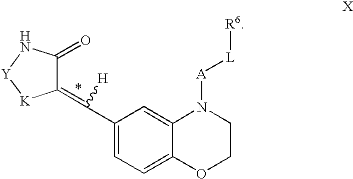

In certain embodiments, R⁶ is H, a C₁₋₉alkyl, a C₂₋₉alkenyl, a C₂₋₉alkynyl, C(C₁-C₅alkyl)(C₁-C₅alkyl), a C₃-C₈cycloalkyl, a phenyl, a naphthalenyl, a 1-naphthalenyl, or a 2-naphthalenyl. In other embodiments, L is absent, a C₁-C₃-alkylene, —CH₂—, —(CH₂)₂—, —CH═CH—, a C₂-C₃-alkenylene, —CH₂—O—, —C₁-C₃-alkyl-O—, —CH₂—O—CH₂—, —C₁-C₃-alkyl-O—C₁-C₃-alkyl, —CH₂—S—, —C₁-C₃-alkyl-S—, or —C₁-C₃-alkyl-S—C₁-C₃-alkyl-. In still other embodiments, A is —C(O)—, —C(O)—O—, or —C(O)—NH—. Examples of compounds of Formula X include, but are not limited to:

4-[2-(3,4-Dichloro-phenyl)-acetyl]-3,4-dihydro-2H-benzo[1,4]oxazine-6-ylmethylene]-2-thioxo-thiazolidin-4-one;

6-(4-Oxo-2-thioxo-thiazolidin-5-ylidenemethyl)-2,3-dihydro-1,4-benzoxazine-4-carboxylic acid phenyl ester;

6-(4-Oxo-2-thioxo-thiazolidin-5-ylidenemethyl)-2,3-dihydro-1,4-benzoxazine-4-carboxylic acid p-tolyl ester;

5-(4-Isobutyryl-3,4-dihydro-2H-1,4-benzoxazin-6-ylmethylene)-2-thioxo-thiazolidin-4-one;

5-(4-Heptanoyl-3,4-dihydro-2H-1,4-benzoxazin-6-ylmethylene)-2-thioxo-thiazolidin-4-one;

5-[4-(3-Cyclopentyl-propionyl)-3,4-dihydro-2H-1,4-benzoxazin-6-ylmethylene]-2-thioxo-thiazolidin-4-one;

5-[4-(3-Phenyl-acryloyl)-3,4-dihydro-2H-1,4-benzoxazin-6-ylmethylene]-2-thioxo-thiazolidin-4-one;

5-[4-(2-Benzyloxy-acetyl)-3,4-dihydro-2H-1,4-benzoxazin-6-ylmethylene]-2-thioxo-thiazolidin-4-one;

5-[4-(2-Phenylsulfanyl-acetyl)-3,4-dihydro-2H-1,4-benzoxazin-6-ylmethylene]-2-thioxo-thiazolidin-4-one;

8-Oxo-8-[6-(4-oxo-2-thioxo-thiazolidin-5-ylidenemethyl)-2,3-dihydro-1,4-benzoxazin-4-yl]-octanoic acid methyl ester;

6-(4-Oxo-2-thioxo-thiazolidin-5-ylidenemethyl)-2,3-dihydro-1,4-benzoxazine-4-carboxylic acid 4-methoxycarbonyl-phenyl ester;

6-(4-Oxo-2-thioxo-thiazolidin-5-ylidenemethyl)-2,3-dihydro-1,4-benzoxazine-4-carboxylic acid (3-trifluoromethyl-phenyl)-amide;

6-(4-Oxo-2-thioxo-thiazolidin-5-ylidenemethyl)-2,3-dihydro-1,4-benzoxazine-4-carboxylic acid phenethyl-amide;

6-(4-Oxo-2-thioxo-thiazolidin-5-ylidenemethyl)-2,3-dihydro-1,4-benzoxazine-4-carboxylic acid cyclopentylamide;

6-(4-Oxo-2-thioxo-thiazolidin-5-ylidenemethyl)-2,3-dihydro-1,4-benzoxazine-4-carboxylic acid naphthalen-1-yl ester;

6-(4-Oxo-2-thioxo-thiazolidin-5-ylidenemethyl)-2,3-dihydro-benzo[1,4]oxazine-4-carboxylic acid (4-chloro-phenyl)-amide;

6-(4-Oxo-2-thioxo-thiazolidin-5-ylidenemethyl)-2,3-dihydro-benzo[1,4]oxazine-4-carboxylic acid (3,4-dichloro-phenyl)-amide;

6-(4-Oxo-2-thioxo-thiazolidin-5-ylidenemethyl)-2,3-dihydro-benzo[1,4]oxazine-4-carboxylic acid (3,5-dimethyl-phenyl)-amide;

6-(4-Oxo-2-thioxo-thiazolidin-5-ylidenemethyl)-2,3-dihydro-benzo[1,4]oxazine-4-carboxylic acid (3-chloro-phenyl)-amide;

5-[4-(3-Methyl-cyclohexanecarbonyl)-3,4-dihydro-2H-benzo[1,4]oxazin-6-ethylene]-2-thioxo-thiazolidin-4-one; and

5-(4-Pentanoyl-3,4-dihydro-2H-1,4-benzoxazin-6-ylmethylene)-2-thioxo-thiazolidin-4-one.

In certain embodiments, W is S, G is C, p is 0, and R⁴, R⁵, R⁷, R⁸, R⁹, R¹⁰, R²⁸, and R³⁰ are H, and the dashed bond between D and E is absent—a compound of Formula XI:

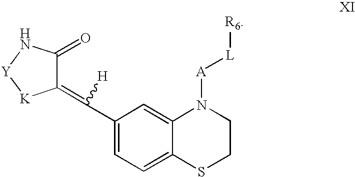

In certain embodiments, R⁶ is H, a C₁₋₉alkyl, a C₂₋₉alkenyl, a C₂₋₉alkynyl, C(C₁-C₅alkyl)(C₁-C₅alkyl), a C₃-C₈cycloalkyl, a phenyl, a naphthalenyl, a 1-naphthalenyl, or a 2-naphthalenyl. In other embodiments, L is absent, a C₁-C₃-alkylene, —CH₂—, —(CH₂)₂—, —CH═CH—, a C₂-C₃-alkenylene, —CH₂—O—, —C₁-C₃-alkyl-O—, —CH₂—O—CH₂—, —C₁-C₃-alkyl-O—C₁-C₃-alkyl, —CH₂—S—, —C₁-C₃-alkyl-S—, or —C₁-C₃-alkyl-S—C₁-C₃-alkyl-. In still other embodiments, A is —C(O)—, —C(O)—O—, or —C(O)—NH—.

In certain embodiments, W is N, R²¹ is methyl, G is C, p is 0, and R⁴, R⁵, R⁷, R⁸, R⁹, R²⁸, and R³⁰ are H, and the dashed bond between D and E is absent—a compound of Formula XII:

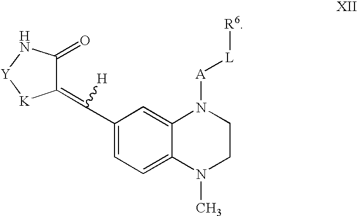

In certain embodiments, R⁶ is H, a C₁₋₉alkyl, a C₂₋₉alkenyl, a C₂₋₉alkynyl, C(C₁-C₅alkyl)(C₁-C₅alkyl), a C₃-C₈cycloalkyl, a phenyl, a naphthalenyl, a 1-naphthalenyl, or a 2-naphthalenyl. In other embodiments, L is absent, a C₁-C₃-alkylene, —CH₂—, —(CH₂)₂—, —CH═CH—, a C₂-C₃-alkenylene, —CH₂—O—, —C₁-C₃-alkyl-O—, —CH₂—O—CH₂—, —C₁-C₃-alkyl-O—C₁-C₃-alkyl, —CH₂—S—, —C₁-C₃-alkyl-S—, or —C₁-C₃-alkyl-S—C₁-C₃-alkyl-. In still other embodiments, A is —C(O)—, —C(O)—O—, or —C(O)—NH—.

In certain embodiments, W is O, G is C, p is 0, R²⁸ is methyl, and R⁴, R⁵, R⁷, R⁸, R⁹, R¹⁰, R²⁸, and R³⁰ are H, and the dashed bond between D and E is absent a compound of Formula XIII:

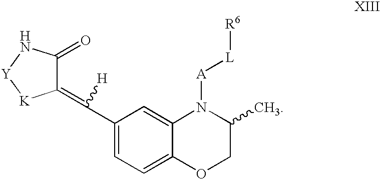

In certain embodiments, R⁶ is H, a C₁₋₉alkyl, a C₂₋₉alkenyl, a C₂₋₉alkynyl, C(C₁-C₅alkyl)(C₁-C₅alkyl), a C₃-C₈cycloalkyl, a phenyl, a naphthalenyl, a 1-naphthalenyl, or a 2-naphthalenyl. In other embodiments, L is absent, a C₁-C₃-alkylene, —CH₂—, —(CH₂)₂—, —CH═CH—, a C₂-C₃-alkenylene, —CH₂—O—, —C₁-C₃-alkyl-O—, —CH₂—O—CH₂—, —C₁-C₃-alkyl-O—C₁-C₃-alkyl, —CH₂—S—, —C₁-C₃-alkyl-S—, or —C₁-C₃-alkyl-S—C₁-C₃-alkyl-. In still other embodiments, A is —C(O)—, —C(O)—O—, or —C(O)—NH—. Examples of compounds of Formula XIII include, but are not limited to:

5-[3-Methyl-4-(-phenyl-methanoyl)-3,4-dihydro-2H-benzo[1,4]oxazine-6-ylmethylene]-2-thioxo-thiazolidin-4-one; and

5-[4-(3,5-Dimethyl-benzyl)-3-methyl-3,4-dihydro-2H-benzo[1,4]oxazine-6-ylmethylene]-2-thioxo-thiazolidin-4-one.

In certain embodiments, W is O, G is C, p is 0, R⁴, R⁷, R⁸, R⁹, R¹⁰, and R²⁸ are H, and the dashed bond between D and E is present—a compound of Formula XIV:

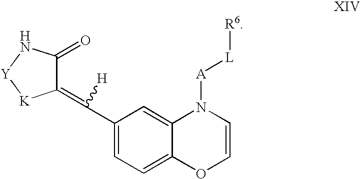

In certain embodiments, R⁶ is H, a C₁₋₉alkyl, a C₂₋₉alkenyl, a C₂₋₉alkynyl, C(C₁-C₅alkyl)(C₁-C₅alkyl), a C₃-C₈cycloalkyl, a phenyl, a naphthalenyl, a 1-naphthalenyl, or a 2-naphthalenyl. In other embodiments, L is absent, a C₁-C₃-alkylene, —CH₂—, —(CH₂)₂—, —CH═CH—, a C₂-C₃-alkenylene, —CH₂—O—, —C₁-C₃-alkyl-O—, —CH₂—O—CH₂—, —C₁-C₃-alkyl-O—C₁-C₃-alkyl, —CH₂—S—, —C₁-C₃-alkyl-S—, or —C₁-C₃-alkyl-S—C₁-C₃-alkyl-. In still other embodiments, A is —C(O)—, —C(O)—O—, or —C(O)—NH—.

In certain embodiments, W is O, G is C, p is 1, and R², R³, R⁴, R⁵, R⁷, R⁸, R⁹, R¹⁰, R²⁸, and R³⁰ are H, and the dashed bond between D and E is absent—a compound of Formula XV:

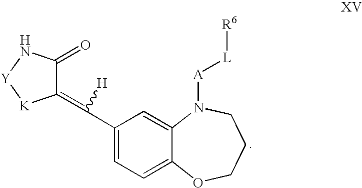

In certain embodiments, R⁶ is H, a C₁₋₉alkyl, a C₂₋₉alkenyl, a C₂₋₉alkynyl, C(C₁-C₅alkyl)(C₁-C₅alkyl), a C₃-C₈cycloalkyl, a phenyl, a naphthalenyl, a 1-naphthalenyl, or a 2-naphthalenyl. In other embodiments, L is absent, a C₁-C₃-alkylene, —CH₂—, —(CH₂)₂—, —CH═CH—, a C₂-C₃-alkenylene, —CH₂—O—, —C₁-C₃-alkyl-O—, —CH₂—O—CH₂—, —C₁-C₃-alkyl-O—C₁-C₃-alkyl, —CH₂—S—, —C₁-C₃-alkyl-S—, or —C₁-C₃-alkyl-S—C₁-C₃-alkyl-. In still other embodiments, A is —C(O)—, —C(O)—O—, or —C(O)—NH—. Examples of compounds of Formula XV include, but are not limited to:

5-[9-(3,5-Dimethoxy-benzyl)-6,7,8,9-tetrahydro-5-oxa-9-aza-benzocyclohepten-2-ylmethylene]-2-thioxo-thiazolidin-4-one; and

5-[9-(3,5-Dimethyl-benzyl)-6,7,8,9-tetrahydro-5-oxa-9-aza-benzocyclohepten-2-ylmethylene]-2-thioxo-thiazolidin-4-one.

In another aspect, the invention provides for pharmaceutical compositions that comprise a therapeutically effective amount of a compound of Formula I; and a pharmaceutically acceptable carrier. In certain embodiments, these compositions are useful in the treatment of a PI3K-mediated disorder or condition. The compounds of the invention can also be combined in a pharmaceutical composition that also comprise compounds that are useful for the treatment of cancer, a thrombolytic disease, heart disease, stroke, an inflammatory disease such as rheumatoid arthritis, or another PI3K-mediated disorder.

In another aspect, the present invention provides for methods of treating a subject suffering from a PI3K-mediated disorder or condition comprising: administering, to a subject suffering from a PI3K-mediated condition or disorder, a pharmaceutical composition comprising a therapeutically effective amount of a compound of Formula I and a pharmaceutically acceptable carrier. In certain embodiments, the PI3K-mediated condition or disorder is selected from the group consisting of: rheumatoid arthritis, ankylosing spondylitis, osteoarthritis, inflammatory diseases, and autoimmune diseases. In other embodiments, the PI3K-mediated condition or disorder is selected from the group consisting of: cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease. In still other embodiments, the PI3K-mediated condition or disorder is selected from the group consisting of: cancer, small cell lung cancer, squamous cell lung carcinoma, glioma, breast cancer, prostate cancer, ovarian cancer, cervical cancer, and leukemia. In yet another embodiment, the PI3K-mediated condition or disorder is selected from the group consisting of: type II diabetes. In still other embodiments, the PI3K-mediated condition or disorder is selected from the group consisting of: respiratory diseases, bronchitis, asthma, and chronic obstructive pulmonary disease. In certain embodiments, the subject is a human.

### Definitions

As used herein, the following terms have the meanings ascribed to them unless specified otherwise.

A “PI3K-mediated disorder or condition” is characterized by the participation of one or more PI3Ks or a PI3P phosphatase, (e.g., PTEN, etc.) in the inception, manifestation of one or more symptoms or disease markers, severity, or progression of a disorder or condition. PI3K-mediated disorders and conditions include, but are not limited to: rheumatoid arthritis, ankylosing spondylitis, osteoarthritis, inflammatory diseases, pulmonary fibrosis, autoimmune diseases, cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, coronary artery disease, cancer, breast cancer, gliobastoma, endometrial carcinoma, hepatocellular carcinoma, colon cancer, lung cancer, melanoma, renal cell carcinoma, thyroid carcinoma, small cell lung cancer, squamous cell lung carcinoma, glioma, breast cancer, prostate cancer, ovarian cancer, cervical cancer, leukemia, cell lymphoma, lymphoproliferative disorders, type II diabetes, respiratory diseases, bronchitis, asthma, and chronic obstructive pulmonary disease.

A PI3K is an enzyme that is able to phosphorylate the 3′-OH of a phosphoinositol to generate PI3P. PI3Ks include, but are not limited to, PI³Kα, PI3Kβ, PI3Kγ, and PI3Kδ. A PI3K typically comprises at least one catalytic subunit (e.g., p110γ), and may further comprise a regulatory subunit (e.g., p101, etc.).

The term “alkyl group” or “alkyl” includes straight and branched carbon chain radicals. The term “alkylene” refers to a diradical of an unsubstituted or substituted alkane. For example, a “C₁₋₆ alkyl” is an alkyl group having from 1 to 6 carbon atoms. Examples of straight-chain alkyl groups include, but are not limited to, methyl, ethyl, n-propyl, n-butyl, n-pentyl, n-hexyl, n-heptyl, n-octyl, n-nonyl, n-decyl, etc. Examples of branched-chain alkyl groups include, but are not limited to, isopropyl, tert-butyl, isobutyl, etc.

Moreover, the term alkyl includes both “unsubstituted alkyls” and “substituted alkyls,” the latter of which refers to alkyl moieties having substituents replacing a hydrogen on one or more carbons (e.g., replacing a hydrogen on 1, 2, 3, 4, 5, or 6 carbons) of the hydrocarbon backbone. Such substituents can include, but are not limited to, C₂-C₆-alkenyl, C₂-C₆-alkynyl, halo, I, Br, Cl, F, —OH, —COOH, sulfhydryl, (C₁-C₆-alkyl)S—, C₁-C₆-alkylsulfinyl, nitro, cyano, trifluoromethyl, —NH₂, ═O, ═S, ═N—CN, ═N—OH, —OCH₂F, —OCHF₂, —OCF₃, —SCF₃, —SO₂—NH₂, C₁-C₆-alkoxy, —C(O)O—(C₁-C₆ alkyl), —O—C(O)—(C₁-C₆ alkyl), —C(O)—NH₂, —C(O)—N(H)—C₁-C₆ alkyl, —C(O)—N(C₁-C₆ alkyl)₂, —OC(O)—NH₂, —C(O)—H, —C(O)—(C₁-C₆ alkyl), —C(S)—(C₁-C₆ alkyl), —NR⁷⁰R⁷², where R⁷⁰ and R⁷² are each independently selected from H, C₁-C₆-alkyl, C₂-C₆-alkenyl, C₂-C₆-alkynyl, and C(O)—C₁-C₆-alkyl.

Alkyl substituents may also include heterocycloalkyl, heteroaryl, and aryl substituents such as, a (C₃-C₈)cycloalkyl, a 3- to 8-membered heterocycloalkyl, phenyl, naphthalenyl, benzyl, phenoxy, naphthalenyl-O—, a 9- to 12-membered bicyclic aryl, a 5-membered heteroaryl, 6-membered heteroaryl, and a 8- to 12-membered bicyclic heteroaryl.

Typical substituted alkyl groups thus are aminomethyl, 2-nitroethyl, 4-cyanobutyl, 2,3-dichloropentyl, and 3-hydroxy-5-carboxyhexyl, 2-aminoethyl, pentachloroethyl, trifluoromethyl, 2-diethylaminoethyl, 2-dimethylaminopropyl, ethoxycarbonylmethyl, methanylsulfanylmethyl, methoxymethyl, 3-hydroxypentyl, 2-carboxybutyl, 4-chlorobutyl, and pentafluoroethyl.

“Alkoxy” refers to the alkyl groups mentioned above bound through oxygen, examples of which include methoxy, ethoxy, isopropoxy, tert-butoxy, and the like. In addition, alkoxy refers to polyethers such as O—(CH₂)₂—O—CH₃, and the like. The term “alkoxy” is intended to include both substituted and unsubstituted alkoxy groups. Alkoxy groups can be substituted on carbon atoms with groups such as those set out above for alkyl. Typical substituted alkoxy groups include aminomethoxy, trifluoromethoxy, 2-diethylaminoethoxy, 2-ethoxycarbonylethoxy, 3-hydroxypropoxy, and the like.

“Alkanoyl” groups are alkyl linked through a carbonyl, e.g., C₁-C₆alkyl-C(O)—. Such groups include formyl, acetyl, propionyl, butyryl, and isobutyryl. The term “alkanoyl” is intended to include both substituted and unsubstituted alkanoyl groups. Alkanoyl groups can be substituted with groups such as those set out above for alkyl.

“Acyl” means an alkyl, cycloalkyl, heteroaryl, heterocycloalkyl, or aryl (Ar) group, etc., bonded through a carbonyl group, i.e., R—C(O)—. For example, acyl includes a C₁-C₆ alkanoyl, including substituted alkanoyl. The term “acyl” is intended to include both substituted and unsubstituted acyl groups. Acyl groups can be substituted with groups such as those set out above for alkyl.

“Halo” includes fluoro, chloro, bromo, and iodo.

“Alkenyl” means straight and branched hydrocarbon radicals having 2 or more carbon atoms and comprising at least one double bond and includes ethenyl, 3-buten-1-yl, 2-ethenylbutyl, 3-hexen-1-yl, and the like. The term “alkenyl” is intended to include both substituted and unsubstituted alkenyl groups. A “C₂-C₆-alkenyl” is an alkenyl group having from from 2 to 6 carbon atoms. Alkenyl groups can be substituted with groups such as those set out above for alkyl. The term “alkenylene” refers to a diradical of a substituted or unsubstituted alkene.

“Alkynyl” means straight and branched hydrocarbon radicals having 2 or more carbon atoms and comprising at least one triple bond and includes ethynyl, 3-butyn-1-yl, propynyl, 2-butyn-1-yl, 3-pentyn-1-yl, and the like. The term “alkynyl” is intended to include both substituted and unsubstituted alkynyl groups. Alkynyl groups can be substituted with groups such as those set out above for alkyl. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms. The term “alkynylene” refers to a diradical of a substituted or unsubstituted alkyne.

“Carbocycle” or “Cycloalkyl” means a mono or bicyclic carbocyclic ring functional group including, but not limited to, cyclopropyl, cyclobutyl, cyclopentyl, cyclohexyl, cycloheptyl, cyclooctyl, cyclononyl, bicyclo[2.2.1]heptanyl, bicyclo[3.2.1]octanyl, and bicyclo[5.2.0]nonanyl; wherein the cycloalkyl group may optionally contain 1 or 2 double bonds (i.e., a cycloalkylenyl) including, but not limited to, cyclopentenyl, cyclohexenyl, and cycloheptenyl. The term “cycloalkyl” is intended to include both substituted and unsubstituted cycloalkyl groups. Cycloalkyl groups and cyclohexyl groups can be substituted with groups such as those set out above for alkyl. Unless otherwise indicated, the term “(C₃-C₈)cycloalkyl” refers to a cycloalkyl group containing from 3 to 8 carbons. Thus, the term “(C₃-C₉)cycloalkyl” encompasses a monocyclic cycloalkyl group containing from 3 to 8 carbons and a bicyclic cycloalkyl group containing from 6 to 8 carbons. Examples of substituted cycloalkyl groups include, but are not limited to, 2-methyl-cyclohexyl, 3-methyl-cyclohexyl, and 4-methyl-cyclohexyl.

The phrase “3- to 8-membered heterocycloalkyl” means a stable cyclic group having carbon atoms and 1 to 3 heteroatoms independently selected from S, N or O, wherein when two O atoms or one O atom and one S atom are present, the two O atoms or one O atom and one S atom are not bonded to each other, respectively. Optionally, a 3- to 8-membered heterocycloalkyl may contain 1 or 2 carbon-carbon or carbon-nitrogen double bonds. Illustrative examples of 3- to 8-membered heterocycloalkyl include aziridin-1-yl, 1-oxa-cyclobutan-2-yl, tetrahydrofuran-3-yl, morpholin-4-yl, 2-thiacyclohex-1-yl, 2-oxo-2-thiacyclohex-1-yl, 2,2-dioxo-2-thiacyclohex-1-yl, and 4-methyl-piperazin-2-yl.

The term “heterocycloalkyl” is intended to include both substituted and unsubstituted heterocycloalkyl groups. Heterocycloalkyl groups can be substituted with 1 to 4 groups such as those set out above for alkyl. Illustrative examples of substituted 3- to 8-membered heterocycloalkyl include 2-hydroxy-aziridin-1-yl, 3-oxo-1-oxacyclobutan-2-yl, 2,2-dimethyl-tetrahydrofuran-3-yl, 3-carboxy-morpholin-4-yl, and 1-cyclopropyl-4-methyl-piperazin-2-yl.

Unless otherwise indicated, the foregoing heterocycloalkyls can be C-attached or N-attached where such is possible and which results in the creation of a stable structure. For example, piperidinyl can be piperidin-1-yl (N-attached) or piperidin-4-yl (C-attached).

Embraced within the term “heterocycloalkyl” are 5 membered rings having one carbon-carbon or one carbon-nitrogen double bond in the ring (e.g., 2-pyrrolinyl, 3-pyrrolinyl, etc.) and 6 membered rings having one carbon-carbon or one carbon-nitrogen double bond in the ring (e.g., dihydro-2H-pyranyl, 1,2,3,4-tetrahydropyridine, 3,4-dihydro-2H-[1,4]oxazine, etc.).

A “3-membered heterocycloalkyl” is a stable 3-membered, monocyclic cycloalkyl ring having 2 carbon atoms and 1 heteroatom selected from the group consisting of: 1 O; 1 S; and 1 N. Illustrative examples of stable 3-membered heterocycloalkyls include oxiranyl, aziridinyl, and thiiranyl.

A “4-membered heterocycloalkyl” is a stable 4-membered, monocyclic cycloalkyl ring having 3 carbon atoms and 1 heteroatom selected from the group consisting of: 1 O; 1 S; and 1 N. Illustrative examples of stable 4-membered heterocycloalkyls include oxetanyl, azetidinyl, and thietanyl.

A “5-membered heterocycloalkyl” is a stable 5-membered, monocyclic cycloalkyl ring having from 1 to 4 carbon atoms and from 1 to 3 heteroatoms selected from the group consisting of: 1 O; 1 S; 1 N; 2 N; 3 N; 1 S and 1 N; 1 S, and 2 N; 1 O and 1 N; and -and 2 N. Illustrative examples of stable 5-membered heterocycloalkyls include tetrahydrofuranyl, dihydrofuranyl, tetrahydrothienyl, dihydrothienyl, imidazolidinyl, oxazolidinyl, imidazolinyl, isoxazolidinyl, pyrrolidinyl, 2-pyrrolinyl, and 3-pyrrolinyl.

A “6-membered heterocycloalkyl” is a stable 6-membered, monocyclic cycloalkyl ring having from 3 to 5 carbon atoms and from 1 to 3 heteroatoms selected from the group consisting of: 1 O; 2 O; 3 O; 1 S; 2 S; 3 S; 1 N; 2 N; 3 N; 1 S, 1 O, and 1 N; 1 S and 1 N; 1 S and 2 N; 1 S and 10; 1 S and 2 O; 1 O and 1 N; and 1 O and 2 N. Illustrative examples of stable 6-membered heterocycloalkyls include tetrahydropyranyl, dihydropyranyl, dioxanyl, 1,3-dioxolanyl, 1,4-dithianyl, hexahydropyrimidine, morpholinyl, piperazinyl, piperidinyl, 2H-pyranyl, 4H-pyranyl, pyrazolidinyl, pyrazolinyl, 1,2,3,6-tetrahydropyridinyl, tetrahydrothiopyranyl, thiomorpholinyl, thioxanyl, and trithianyl.

A “7-membered heterocycloalkyl” is a stable 7-membered, monocyclic cycloalkyl ring having from 5 or 6 carbon atoms and from 1 to 3 heteroatoms selected from the group consisting of: 1 O; 2 O; 1 S; 2 S; 1 N; 2 N; 1 S, 1 O, and 1 N; 1 S and 1 N; 1 S and 2 N; 1 S and 1 O; 1 S and 2 O; 1 O and 1 N; and 1 O and 2 N. Illustrative examples of stable 7-membered heterocycloalkyls include azepanyl, 2,3,4,5-tetrahydro-1H-azepinyl, oxepanyl, 2,3,4,5-tetrahydro-1H-oxepinyl, thiepanyl, and 2,3,4,5-tetrahydro-1H-thiepinyl.

An “8-membered heterocycloalkyl” is a stable 8-membered, monocyclic cycloalkyl ring having from 5 to 7 carbon atoms and from 1 to 3 heteroatoms selected from the group consisting of: 1 O; 2 O; 3 O; 1 S; 2 S; 3 S; 1 N; 2 N; 3 N; 1 S, 1 O, and 1 N; 1 S and 1 N; 1 S and 2 N; 1 S and 1 O; 1 S and 2 O; 1 O and 1 N; and 1 O and 2 N. Illustrative examples of stable 8-membered heterocycloalkyls include azocanyl, thiocanyl, oxocanyl, 3,4,5,6-tetrahydro-2H-oxocinyl, etc.

The term “3- to 8-membered heterocycloalkyl” includes saturated and unsaturated “3- to 8-membered heterocycloalkyls.” “3- to 8-membered heterocycloalkyls” may be substituted as set out above for alkyl.

The term “6- to 11-membered bicyclic heterocycloalkyl” refers to a stable ring structure which is either saturated or unsaturated, and which is the result of the fusion of a 5-, 6-, or 7-membered heterocycloalkyl to a 3-, 4-, 5-, 6-, or 7-membered heterocycloalkyl; or a 5-, 6-, or 7-membered heterocycloalkyl to a C₃₋₇-cycloalkyl, wherein the fusion junctions are at adjacent ring atoms. The term “6- to 11-membered bicyclic heterocycloalkyl” includes saturated and unsaturated “6- to 11-membered bicyclic heterocycloalkyls.” “6- to 11-membered bicyclic heterocycloalkyls” may be substituted as set out above for alkyl. Examples of “6- to 11-membered bicyclic heterocycloalkyls” include 3-azabicyclo[3.1.0]hexanyl, and 3-azabicyclo[4.1.0]-heptanyl.

The term “6- to 9-membered bridged bicyclic heterocycloalkyl” refers to a stable ring structure which is either saturated or unsaturated, and which is the result of the fusion of 5-, 6-, or 7-membered heterocycloalkyl to a 3-, 4-, or 5-membered heterocycloalkyl; or a 5-, 6-, or 7-membered heterocycloalkyl to a C₅₋₇-cycloalkyl, wherein the fusion junctions have 1 to 3 intervening ring atoms. The term “6- to 9-membered bridged bicyclic heterocycloalkyl” includes saturated and unsaturated “6- to 9-membered bridged bicyclic heterocycloalkyls.” “6- to 9-membered bridged bicyclic heterocycloalkyls” may be substituted as set out above for alkyl. Examples of “6- to 9-membered bridged bicyclic heterocycloalkyls” include 3-azabicyclo[4.2.1]nonanyl and 7-azabicyclo[2.2.1]heptanyl.

An aryl group is an aromatic hydrocarbon radical. Furthermore, the term “aryl” includes multicyclic aryl groups, bicyclic, e.g., naphthyl. Typical aryl groups include phenyl, and naphthyl. Phenyl may be unsubstituted or substituted at one or more positions with a substituent such as, but not limited to, those substituents described above for alkyl. Typical substituted phenyl groups include, but are not limited to, 3-chlorophenyl, 2,6-dibromophenyl, 2,4,6-tribromophenyl, 2,6-dichlorophenyl, 4-trifluoromethylphenyl, 3-amino-4-nitrophenyl, 3,5-dihydroxyphenyl, 3-methyl-phenyl, 4-methyl-phenyl, 3,5-dimethyl-phenyl, 3,4,5-trimethoxy-phenyl, 3,5-dimethoxy-phenyl, 3,4-dimethoxy-phenyl, 3-methoxy-phenyl, 4-methoxy-phenyl, 4-tert-butyl-phenyl, 4-hexyl-phenyl, 4-cyano-phenyl, 3,5-di-triflouromethyl-phenyl, 3,5-difluoro-phenyl, 4-chloro-phenyl, 3-trifluoromethyl-phenyl, 4-methoxycarbonyl-phenyl, 2-trifluoromethoxy-phenyl, 3,5-dichloro-phenyl, 2-methoxy-5-methyl-phenyl, 2-fluoro-5-methyl-phenyl, 4-phenoxy-phenyl, 4-chloro-2-trifluoromethyl-phenyl, and the like. Naphthalenyl may be unsubstituted or substituted at one or more positions with a substituent such as, but not limited to, those substituents described above for alkyl. The term “aryl” is intended to include both substituted and unsubstituted phenyl groups.

A “9- to 12-membered bicyclic aryl” is a stable ring structure formed by the fusion of a benzene ring to:

(1) a C₅₋₈ monocyclic cycloalkyl (e.g., indanyl; 1,2,3,4-tetrahydro-naphthalenyl; 6,7,8,9-tetrahydro-5H-benzocycloheptenyl, etc.);

(2) a 5- to 7-membered heterocycloalkyl (e.g., benzoxazine, benzthiazine, chromanyl, 1,2,3,4-tetrahydro-quinolinyl, etc.); or

(3) another benzene ring (e.g., naphthalenyl);

wherein the fusion junctions are at adjacent carbons on the benzene ring.

A “5-membered heteroaryl” is a stable 5-membered, monocyclic, aromatic ring radial having from 1 to 4 carbon atoms and from 1 to 4 heteroatoms selected from the group consisting of: 1 O; 1 S; 1 N; 2 N; 3 N; 4 N; 1 S and 1 N; 1 S and 2 N; 1 O and 1 N; and 1 O and 2 N. Illustrative examples of stable 5-membered heteroaryls include, but are not limited to, furanyl, 2-furanyl, 3-furanyl, imidazolyl, isoxazolyl, isothiazolyl, oxadiazolyl, oxazolyl, pyridinyl, 2-, 3-, or 4-pyridinyl, pyrimidinyl, 2-, 4-, or 5-pyrimidinyl, pyrazolyl, pyrrolyl, 2- or 3-pyrrolyl, pyrazinyl, pyridazinyl, 3- or 4-pyridazinyl, 2-pyrazinyl, thienyl, 2-thienyl, 3-thienyl, tetrazolyl, thiazolyl, thiadiazolyl, triazinyl and triazolyl.

A “6-membered heteroaryl” is a stable 6-membered, monocyclic, aromatic ring radical having from 3 to 5 carbon atoms and from 1 to 3 heteroatoms selected from the group consisting of: 1 N; 2 N; and 3 N. Illustrative examples of stable 6-membered heteroaryl include pyridin-2-yl, pyridin-4-yl, pyrimidin-2-yl, pyridazin-4-yl, and pyrazin-2-yl.

An “8- to 12-membered bicyclic heteroaryl” is a stable ring structure formed by the fusion of 5- or 6-membered heteroaryl to:

(1) an independently selected 5-membered heteroaryl;

(2) an independently selected 6-membered heteroaryl (e.g., naphthyridinyl, pteridinyl, phthalazinyl, purinyl, etc.);

(3) a C₅₋₈ monocyclic cycloalkyl;

(4) a 5- to 7-membered heterocycloalkyl; or

(5) a benzene ring (e.g., benzimidazolyl, benzofuranyl, benzofurazanyl, 2H-1-benzopyranyl, benzothiadiazine, benzothiazinyl, benzothiazolyl, benzothiophenyl, benzoxazolyl, cinnolinyl, furopyridinyl, indolinyl, indolizinyl, indolyl, or 2-, 3-, 4-, 5-, 6-, or 7-indolyl, 3H-indolyl, quinazolinyl, quinoxalinyl, isoindolyl, and isoquinolinyl), wherein the fusion junctions are at adjacent ring atoms. The fusion junctions may be at a nitrogen (e.g., indolizine) or at carbon atoms in a 5- or 6-membered heteroaryl.

A heteroaryl can also include ring systems substituted on ring carbons with one or more —OH functional groups (which may further tautomerize to give a ring C═O group) and or substituted on a ring sulfur atom by 1 or 2 oxygen atoms to give S═O, or SO₂ groups, respectively.

The phrase “pharmaceutical composition” refers to a composition suitable for administration in medical or veterinary use.

The phrase “therapeutically effective amount” means an amount of a compound, or a pharmaceutically acceptable salt thereof, sufficient to inhibit, halt, or cause an improvement in the disorder or condition being treated in a particular subject or subject population. For example in a human or other mammal, a therapeutically effective amount can be determined experimentally in a laboratory or clinical setting, or may be the amount required by the guidelines of the United States Food and Drug Administration, or equivalent foreign agency, for the particular disease and subject being treated.

It should be appreciated that determination of proper dosage forms, dosage amounts, and routes of administration is within the level of ordinary skill in the pharmaceutical and medical arts, and is described below.

Some of the compounds in the present invention may exist as stereoisomers, including enantiomers, diastereomers, and geometric isomers. Geometric isomers include compounds of the present invention that have alkenyl groups, which may exist as entgegen or zusammen conformations, in which case all geometric forms thereof, both entgegen and zusammen, cis and trans, and mixtures thereof, are within the scope of the present invention. Some compounds of the present invention have cycloalkyl groups, which may be substituted at more than one carbon atom, in which case all geometric forms thereof, both cis and trans, and mixtures thereof, are within the scope of the present invention. All of these forms, including (R), (S), epimers, diastereomers, cis, trans, syn, anti, (E), (Z), tautomers, and mixtures thereof, are contemplated in the compounds of the present invention.

The compounds to be used in the present invention can exist in unsolvated forms as well as solvated forms, including hydrated forms. In general, the solvated forms, including hydrated forms, are equivalent to unsolvated forms and are intended to be encompassed within the scope of the present invention.

The compounds of the present invention (e.g., compounds of Formula I) are capable of further forming both pharmaceutically acceptable salts, including but not limited to acid addition and/or base salts. This invention also provides pharmaceutical compositions comprising a compound of Formula I together with a pharmaceutically acceptable carrier, diluent, or excipient therefor. All of these forms can be used in the method of the present invention.

Pharmaceutically acceptable acid addition salts of the compounds of Formula I include salts derived from inorganic acids such as hydrochloric, nitric, phosphoric, sulfuric, hydrobromic, hydriodic, phosphorus, and the like, as well as the salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are the salts of amino acids such as arginate, gluconate, galacturonate, and the like; see, for example, Berge et al., “Pharmaceutical Salts,” J. of Pharmaceutical Science, 1977;66:1-19.

The acid addition salts of the basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metal hydroxides, or of organic amines. Examples of metals used as cations are sodium, potassium, magnesium, calcium, and the like. Examples of suitable amines are N,N′-dibenzylethylenediamine, chloroprocaine, choline, diethanolamine, ethylenediamine (ethane-1,2-diamine), N-methylglucamine, and procaine; see, for example, Berge et al., supra., 1977.

The base addition salts of acidic compounds are prepared by contacting the free acid form with a sufficient amount of the desired base to produce the salt in the conventional manner. The free acid form may be regenerated by contacting the salt form with an acid and isolating the free acid in a conventional manner. The free acid forms differ from their respective salt forms somewhat in certain physical properties such as solubility in polar solvents, but otherwise the salts are equivalent to their respective free acid for purposes of the present invention.

“Cancer cells,” “transformed” cells, or “transformation” in tissue culture, refers to spontaneous or induced phenotypic changes that do not necessarily involve the uptake of new genetic material. Although transformation can arise from infection with a transforming virus and incorporation of new genomic DNA, or uptake of exogenous DNA, it can also arise spontaneously or following exposure to a carcinogen, thereby mutating an endogenous gene. Transformation is associated with phenotypic changes, such as immortalization of cells, aberrant growth control, and/or malignancy (see, Freshney, Culture of Animal Cells: A Manual of Basic Technique, 4th ed. Wiley-Liss, Inc., 2000).

The term “subject” refers to a member of the class Mammalia. Examples of mammals include, without limitation, humans, primates, chimpanzees, rodents, mice, rats, rabbits, horses, livestock, dogs, cats, sheep, and cows.

The term “treatment” includes the acute or prophylactic diminishment or alleviation of at least one symptom or characteristic associated or caused by the disorder being treated. For example, treatment can include diminishment of several symptoms of a disorder or complete eradication of a disorder.

The term “administering” refers to the method of contacting a compound with a subject. Modes of “administering” may include but are not limited to, methods that involve contacting the compound intravenously, intraperitoneally, intranasally, transdermally, topically, via implantation, subcutaneously, parentally, intramuscularly, orally, systemically, and via adsorption.

### DETAILED DESCRIPTION OF THE INVENTION

I. Introduction

The present invention relates to compounds of Formula I and pharmaceutically acceptable salts thereof:

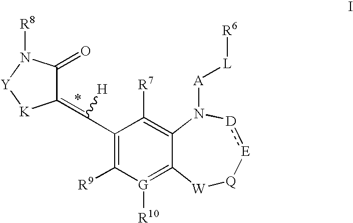

wherein W, Q, E, D, A, L, R⁶, R⁷, R⁸, Y, K, R⁹, R¹⁰, G, the dashed bond between D and E, and the double bond denoted “*” have any of the values defined therefore in the specification. Compounds of Formula I, and pharmaceutical compositions thereof, are useful as agents in the treatment of diseases and conditions, including inflammatory diseases, cardiovascular diseases, and cancers. Also disclosed are pharmaceutical compositions comprising one or more compounds of Formula I, processes for preparing compounds of Formula I, and intermediates useful for preparing compounds of Formula I. In particular, compounds of the present invention are useful for the treatment of a PI3K-mediated disorder or condition.

II. Preparation of Compounds

Compounds of the present invention (e.g., compounds of Formula I) can be prepared by applying synthetic methodology known in the art and synthetic methodology outlined in the schemes set forth below.

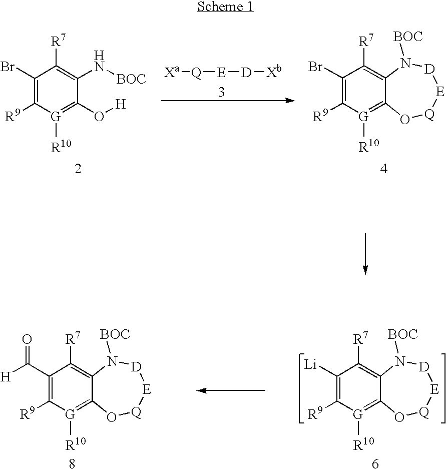

In Scheme 1, an appropriately substituted BOC protected-bromo-aminophenol (e.g., 4-bromo-2-aminophenol) or BOC-protected bromo-amino-pyridin-ol (e.g., 5-bromo-3-amino-pyridin-2-ol) 2 (e.g., 4-bromo-N-(tert-butoxycarbonyl)-2-aminophenol) is reacted with a dihalogenated straight or branched chain alkane 3 (X^{a}-Q-E-D-X^{b}) to yield 4 (see e.g., Buon et al. (2000) Tetrahedron 56: 605-614). X^{a} and X^{b} are independently selected from Cl, I, F, and Br. Examples of 7 include, but are not limited to, 1,3-dibromoethane and 1,3-dibromopropane. The reaction is carried out in the presence of a non-nucleophilic organic base (e.g., triethylamine) or an inorganic base (e.g., Na₂CO₃, K₂CO₃, NaH, CsCO₃, etc.), optionally in the presence of a phase transfer reagent (e.g., benzyl triethylammonium chloride) in a solvent such as 3-pentanone. Compounds of formula 2 can be prepared from an appropriately substituted bromo-aminophenol or bromo-amino-pyridin-ol using procedures such as those described in Buon et al. (2000) Tetrahedron 56: 605-614. Those of skill in the art will recognized that a variety of amine protecting groups in addition to BOC (t-butyl-O—C(O)—) can be used in Scheme 1 (see e.g., Greene and Wuts, Protective Groups in Organic Synthesis, 2nd ed., Chapter 7 (John Wiley & Sons, Inc., 1991)).

The compound 4 is then further reacted with an alkyl lithium reagent (e.g., t-butyl-Li, sec-butyl-Li, etc.) at a temperature from about −100° C. to about 0° C. (e.g., −78° C.) in an aprotic solvent (e.g., hexanes, THF (tetrahydrofuran), ether, etc.) to allow a bromine-lithium exchange to yield 6 in situ. The compound 6 is then reacted with a dialkylformamide such as DMF (dimethylformamide) to give 8.

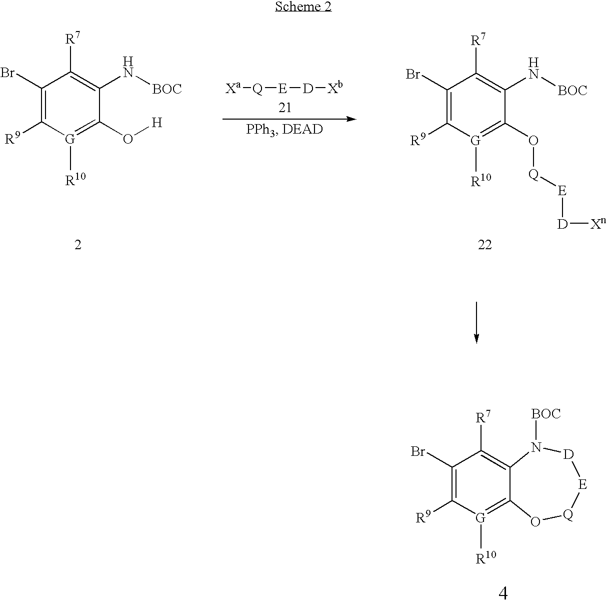

Alternatively, 2 can be reacted with a monohalogenated alkyl alcohol 21 (e.g., 2-bromo-propan-1-ol) as illustrated in Scheme 2 under Mitsunobu conditions (e.g., PPh₃ (triphenylphosphine) and DEAD (diethyl azodicarboxylate) in a solvent such as dichloromethane to arrive at 22. Examples of 21 include straight-chain alkyl alcohols (e.g., HO-Q-E-D-Xⁿ, 2-bromo-ethanol, etc.) and branched-chain alkyl alcohols (e.g., HO-Q-E-CH(CH₃)-Xⁿ, 2-bromo-propan-1-ol, etc). Xⁿ is selected from Cl, I, F, and Br. Compound 22 is then cyclized using conditions such as those described above in Scheme 1 for the condensation and cyclization of 2 with 3 to give 4.

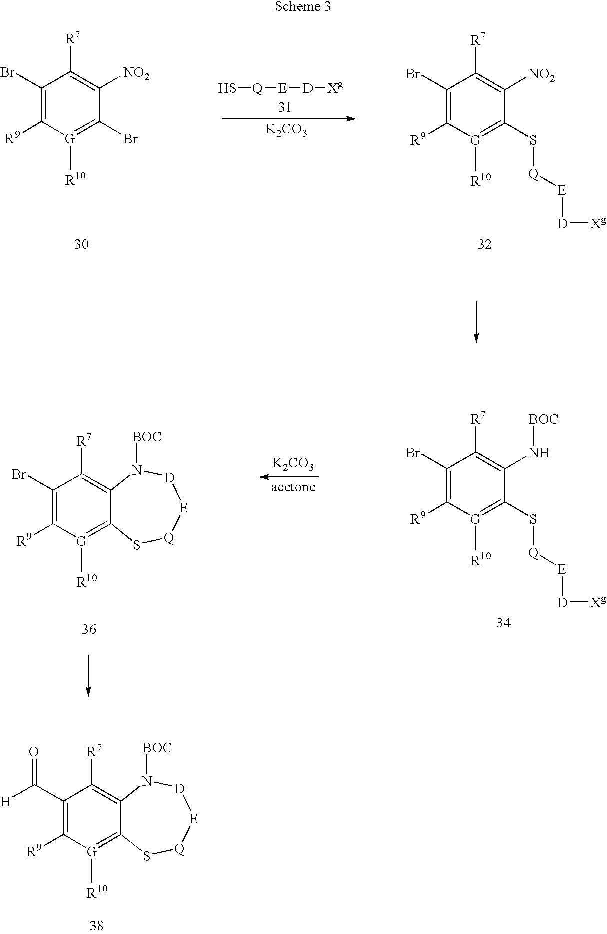

In Scheme 3, an appropriately substituted dibromo-nitro-benzene (e.g., 1,4-dibromo-2-nitro-benzene) or dibromo-nitro-pyridine (e.g., 2,5-dibromo-3-nitro-pyridine) 30 is reacted with an monohalogenated alkyl thiol 31 (HS-Q-E-D-X^{g}), such as 2-chloro-ethanethiol, and potassium carbonate in acetone to form 32 (e.g., 4-bromo-1-(2-chloro-ethylsulfanyl)-2-nitro-benzene). X^{g} is Cl, Br, I, or F.

The nitro group of 32 is then reduced to an amine by a reducing agent such as borane, zinc metal in acid, dithionate, tin metal in acid, or with hydrogen gas at a suitable pressure (e.g., 69 psi) and a catalyst (e.g., Raney Nickel). The amine is then protected as a BOC (t-butyl-O—C(O)—) derivative with a reagent such as di-tert-butyl-dicarbonate to give 34 (e.g., 4-bromo-N-(tert-butoxycarbonyl)-2-aminophenol). The compound 34 is then reacted with an inorganic base (e.g., potassium carbonate) in a solvent such as acetone to provide 36. The compound 36 is further reacted as in Scheme 1 with an alkyl lithium reagent followed by a dialkylformamide to yield 38.

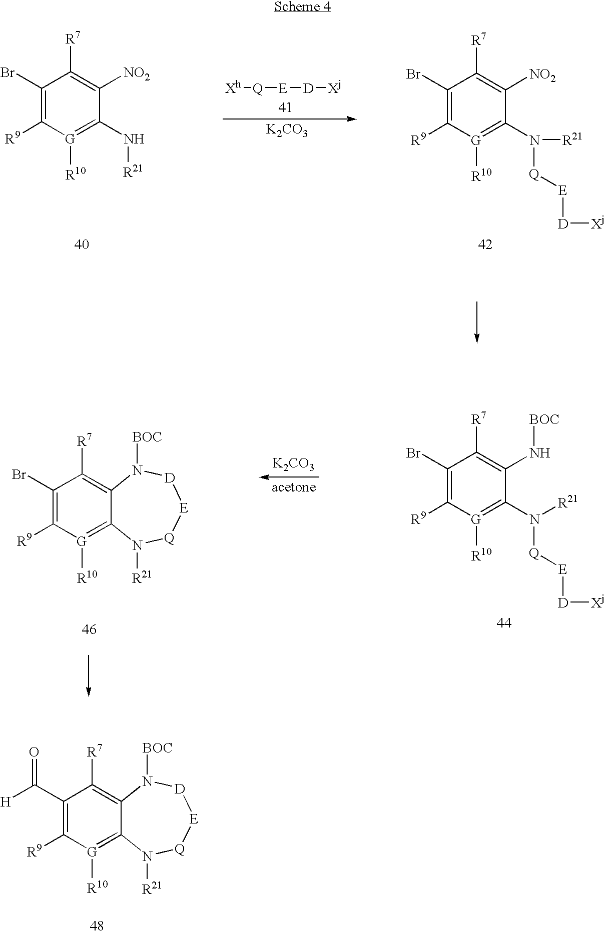

In Scheme 4,40, an appropriately substituted 4-bromo-2-nitro-phenylamine or 5-bromo-3-nitro-pyridin-2-ylamine, is reacted with an dihalogenated straight or branched chain alkane 41 (X^{h}-Q-E-D-X^{j}), such as 1,3-dibromoethane, in the presence of potassium carbonate in acetone to form 42 (e.g., 4-bromo-1-(2-chloro-ethylsulfanyl)-2-nitro-benzene). The reaction can also be carried out using the reaction conditions of Scheme 1 for the transformation of 2 to 4. X^{g} is Cl, Br, I, or F.

The nitro group of 42 is then reduced to an amine and protected with a BOC group in a fashion similar to Scheme 3 to yield 44. 44 is then reacted with an inorganic base as in Scheme 3 to provide 46. 46 is then treated with an alkyl lithium reagent followed by a dialkylformamide to yield 48 as in Scheme 1.

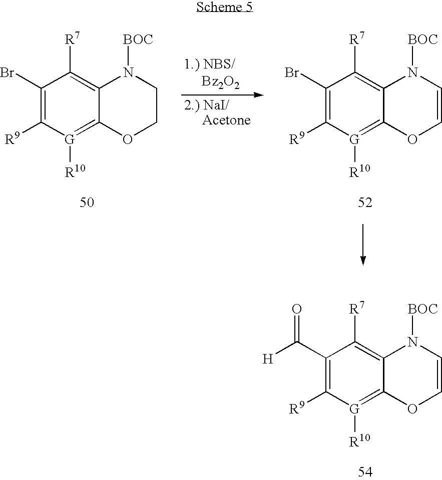

In Scheme 5, a route for the synthesis of a 4H-benzo[1,4]oxazine 54 is depicted. The compound 50, in dry carbon tetrachloride, is first treated with a brominating agent such as N-bromosuccinimide (NBS) and a catalytic amount of benzoyl peroxide (Bz₂O₂) using methods such as those described in Buon et al. (2000) Tetrahedron 56: 605-614. The resulting reaction product is then treated with sodium iodide (NaI) in acetone as described in Buon et al. (2000) Tetrahedron 56: 605-614 to obtain 52. 52 is then treated with an alkyl lithium reagent followed by a dialkylformamide as in Scheme 1 to provide 54.

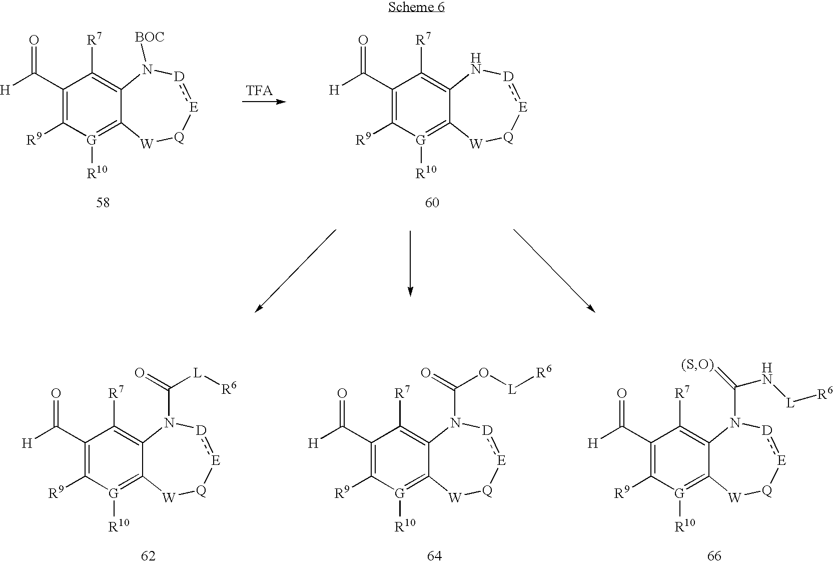

As set out in Scheme 6, the BOC group of 58 (e.g., 8, 38, 46, 54, etc.) is removed with acid (e.g., TFA (trifluoroacetic acid), HCl, HBr, etc.) to give the amine 60. Compounds such as 60 can then be reacted with an acylhalide, (e.g., R⁶-L-C(O)-X^{c}, where X^{c} is Br, I, F, or Cl) to form 62 (e.g., 5-[4-(1-phenyl-methanoyl)-3,4-dihydro-2H-benzo[1,4]oxazine-6-ylmethylene]-2-thioxo-thiazolidin-4-one). Examples of acylhalides include, but are not limited to, benzoyl chloride, furan-2-carbonyl chloride, cyclohexanecarbonyl chloride, 4-methanesulfonyl-benzoyl chloride, isonicotinoyl chloride, and nicotinoyl chloride. Also, sulfonyl halides (e.g., benzenesulfonyl chloride) can be reacted to with 60 to form the corresponding sulfonyl benzoxazine derivative 68:

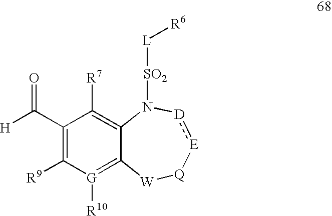

Alternatively, 60 can be reacted with an isocyanate (e.g., R⁶-L-N═C═O) or with an isothiocyanate (e.g., R⁶-L-N═C═S) to form 66 (e.g., 6-(4-Oxo-2-thioxo-thiazolidin-5-ylidenemethyl)-2,3-dihydro-benzo[1,4]oxazine-4-carboxylic acid (3,4-dimethoxy-phenyl)-amide). Examples of isocyanates that can be used in this reaction include, but are not limited to, phenyl isocyanate (isocyanato-benzene), 4-isocyanato-1,2-dimethoxy-benzene, 1,3-dichloro-5-isocyanato-benzene, 1-chloro-4-isocyanato-benzene, 1,2-dichloro-4-isocyanato-benzene, 1,3-dimethyl-5-isocyanato-benzene, and 1-chloro-3-isocyanato-benzene.

In addition, the carbamate 64 (e.g., 6-(4-Oxo-2-thioxo-thiazolidin-5-ylidenemethyl)-2,3-dihydro-1,4-benzoxazine-4-carboxylic acid phenyl ester) can be provided by reacting a haloformate (e.g., R⁶-L-O—C(O)—X^{d}, where X^{d} is Br, I, F, or Cl) with 60. In certain embodiments, chloroformates are preferred. Examples of chloroformates include, but are not limited to phenyl chloroformate, 4-methoxycarbonyl-phenyl chloroformate, naphthalenyl chloroformate, and p-tolyl chloroformate.

The reaction of 60 to form 62, 64, 66, or 68, can be carried out in the presence of an aprotic solvent such as acetonitrile, dichloromethane or 1,2-dichloroethane and a non-nucleophilic organic base such as triethylamine or an inorganic base such as sodium carbonate at room temperature.

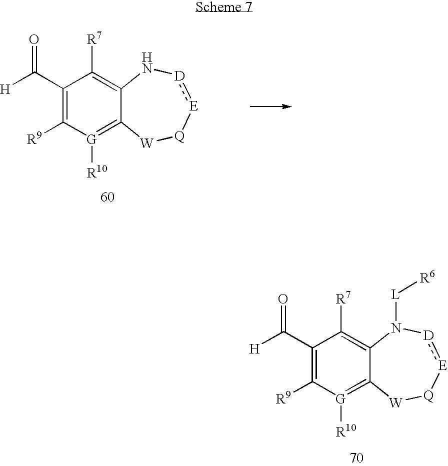

The compound 70 (e.g., 4-(3,5-dimethyl-benzyl)-3,4-dihydro-2H-benzo[1,4]oxazine-6-ylmethylene]-2-thioxo-thiazolidin-4-one) can be provided as set out in Scheme 7 by reacting 60 with an alkylhalide, arylhalide, heteroarylhalide, cycloalkylhalide, etc. (e.g., R⁶-L-X^{f}, where X^{f} is Br, I, F, or Cl) in the presence of a non-nucleophilic base such as sodium hydride, triethylamine, potassium carbonate, cesium carbonate or 2-tert-butylimino-2-diethylamino-1,3-dimethyl-perhydro-1,3,2-diazo-phosphorine on polystyrene in an organic solvent such as THF, DMF, or acetonitrile. Examples of compounds of R⁶-L-X^{f} include, but are not limited to, 3,5-dimethylbenzyl bromide, 3,5-di-tert-butyl-benzyl bromide, and (2-bromoethyl)-benzene.

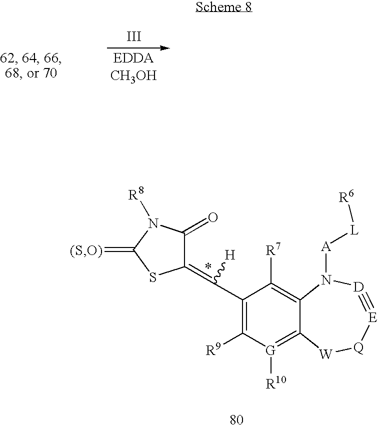

In Scheme 8, 62, 64, 66, 68, or 70 is reacted with a compound of formula III containing an activated methylene group, for example: a rhodanine (e.g., rhodanine, rhodanine-3-acetic acid, 3-phenyl rhodanine, etc.) or a thiazolidinedione (e.g,. thiazolidinedione, etc.), in the presence of an organic base, such as ethylenediamine diacetate (EDDA), diisopropylethylamine, sodium acetate or pyridine, in the presence of acetic acid and methanol to form a compound of 80. Alternatively, in a Knoevenagel condensation of the active methylene of III with 62, 64, 66, 68, or 70 can be carried out to yield 80, using ammonium acetate in toluene and heating to a high temperature (e.g., 110° C.), according to procedures such as those described in Lee and Sun (2000) Tetrahedron Lett. 41: 5729-5732.

Compounds of formula III are defined herein as a compound having the following structure:

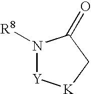

where Y is C(O) or C(S), and K is S. Examples of compounds of formula III include rhodanine and rhodanine derivatives:

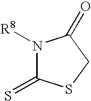

and thiazolidine dione and thiazolidine dione derivatives:

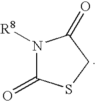

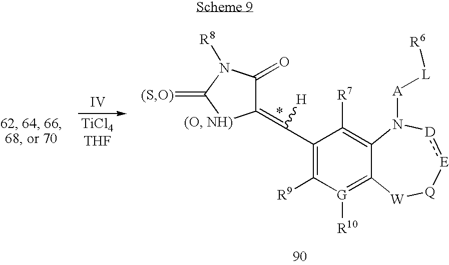

In Scheme 9, 62, 64, 66, 68, or 70 is reacted with a compound of formula IV, such as a imidazolidine-2,4-dione or a 2-thioxo-oxazolidin-4-one, in the presence of titanium tetrachloride (TiCl₄) and pyridine in THF to form a compound of formula 90. Compounds of formula IV are defined herein in a compound having the following structure:

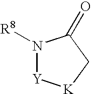

where Y is C(O) or C(S), and K is O or NH. Examples of compounds of formula IV include imidazolidine-2,4-dione and imidazolidine-2,4-dione derivatives:

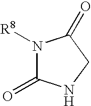

and 2-thioxo-oxazolidin-4-one and 2-thioxo-oxazolidin-4-one derivatives:

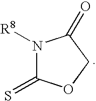

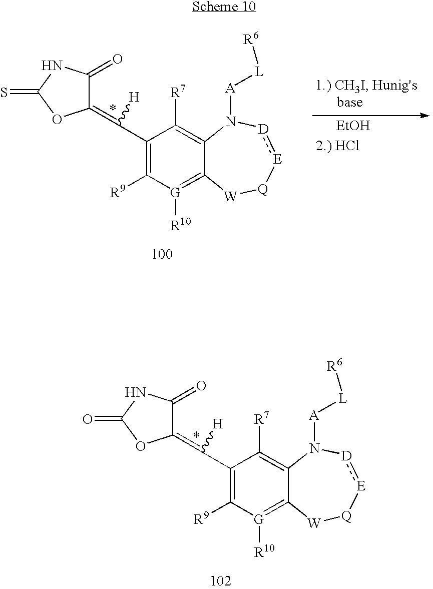

A 2-thioxo-oxazolidin-4-one derivative obtained using Scheme 9, such as 100, can be converted to an oxazolidine-2,4-dione 102 with iodomethane and Hunig's base in ethanol, followed by hydrolysis, e.g., with concentrated HCl, as depicted in Scheme 10.

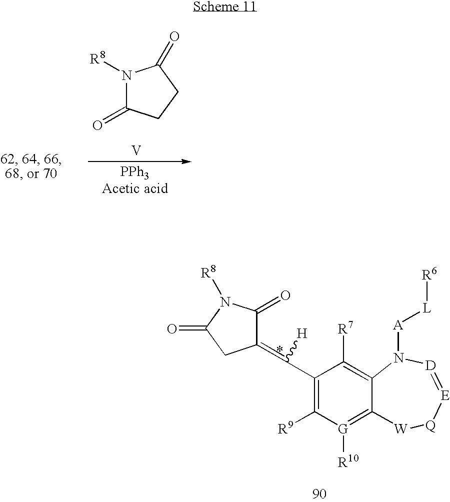

In Scheme 11, 62, 64, 66, 68, or 70 is reacted with a compound of formula V, such as a pyrrolidine-2,5-dione in the presence of triphenylphosphine (PPh₃) and acetic acid to form a compound of the formula 90. Compounds of formula V are defined herein in a compound having the following structure:

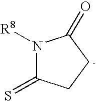

III. Evaluation of Compounds

Compounds of the present invention (e.g., compounds of Formula I and pharmaceutically acceptable salts thereof) can be assayed for their ability to inhibit a PI3K. Examples of these assays are set out below and include in vitro and in vivo assays of PI3K activity.

In certain embodiments of the present invention are compounds that selectively inhibit one or more PI3Ks as compared to one or more enzymes including, but not limited to, a cyclic nucleotide dependent protein kinase, PDGF, a tyrosine kinase, a MAP kinase, a MAP kinase kinase, a MEKK, a cyclin-dependent protein kinase. In other embodiments of the invention are compounds that selectively inhibit one PI3K as compared to another PI3K. For example, in certain embodiments, compounds of the present invention display the ability to selectively inhibit PI3Kγ as compared to PI3Kα or PI3Kβ. A compound selectively inhibits a first enzyme as compared to a second enzyme, when the IC₅₀ of the compound towards the first enzyme is less than the IC₅₀ of the compound towards the second compound. The IC₅₀ can be measured, for example, in an in vitro PI3K assay.

In presently preferred embodiments, compounds of the present invention can be assessed for their ability to inhibit PI3Kactivity in an in vitro or an in vivo assay (see below).

PI3K assays are carried out in the presence or absence of a PI3K inhibitory compound, and the amount of enzyme activity is compared for a determination of inhibitory activity of the PI3K inhibitory compound.

Samples that do not contain a PI3K inhibitory compound are assigned a relative PI3K activity value of 100. Inhibition of PI3K activity is achieved when the PI3K activity in the presence of a PI3K inhibitory compound is less than the control sample (i.e., no inhibitory compound). The IC₅₀ of a compound is the concentration of compound that exhibits 50% of the control sample activity. In certain embodiments, compounds of the present invention have an IC₅₀ of less than about 100 μM. In other embodiments, compounds of the present invention have an IC₅₀ of about 1 μM or less. In still other embodiments, compounds of the present invention have an IC₅₀ of about 200 nM or less.

PI3Kγ assays have been described in the art (see e.g., Leopoldt et al. J. Biol. Chem., 1998;273:7024-7029). Typically, a sample containing a complex of p101 and 110γ protein are combined with Gβ and Gγ proteins (e.g., G protein β₁/γ₂ subunits). Radiolabeled ATP (e.g., γ-³²P-ATP) is then added to this mixture. The lipid substrates are formed by creating PIP₂ containing lipid micelles. The reactions are then started by adding the lipid and enzyme mixtures and are stopped with the addition of H₃PO₄. The lipid products are then transferred to a glass fiber filter plate, and washed with H₃PO₄ several times. The presence of radioactive lipid product (PIP₃) can be measured using radiometric methods that are well-known in the art.

The activity of growth factor regulated PI3Ks can also be measured using a lipid kinase assay. For example, PI3Kα can be assayed using samples that contain a regulatory and a catalytic subunit. An activating peptide (e.g., pY peptide, SynPep Corp.) is added to the sample with radiolabeled ATP. PIP₂ containing lipid micelles are then added to the sample to start the reaction. The reactions are worked up and analyzed as described for the PI3Kγ assay just described. Assays can also be carried out using cellular extracts (Susa et al. J. Biol. Chem., 1992;267:22951-22956).

IV. Pharmaceutical Compositions

The present invention also provides pharmaceutical compositions comprising a pharmaceutically acceptable carrier or excipient and a compound of the present invention (e.g., a compound of Formula I, or a pharmaceutically acceptable salt thereof). A compound of the present invention can be formulated as a pharmaceutical composition in the form of a syrup, an elixir, a suspension, a powder, a granule, a tablet, a capsule, a lozenge, a troche, an aqueous solution, a cream, an ointment, a lotion, a gel, an emulsion, etc. Preferably, a compound of the present invention will cause a decrease in symptoms or a disease indicia associated with a PI3K-mediated disorder as measured quantitatively or qualitatively.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired.

The powders and tablets contain from 1% to 95% (w/w) of the active compound. In certain embodiments, the active compound ranges from 5% to 70% (w/w). Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 1000 mg, preferably 1.0 mg to 100 mg, or from 1% to 95% (w/w) of a unit dose, according to the particular application and the potency of the active component. The composition can, if desired, also contain other compatible therapeutic agents.

Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington: The Science and Practice of Pharmacy, 20th ed., Gennaro et al. Eds., Lippincott Williams and Wilkins, 2000).

A compound of the present invention, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

The dose administered to a subject, in the context of the present invention should be sufficient to effect a beneficial therapeutic response in the subject over time. The dose will be determined by the efficacy of the particular compound employed and the condition of the subject, as well as the body weight or surface area of the subject to be treated. The size of the dose also will be determined by the existence, nature, and extent of any adverse side-effects that accompany the administration of a particular compound in a particular subject. In determining the effective amount of the compound to be administered in the treatment or prophylaxis of the disorder being treated, the physician can evaluate factors such as the circulating plasma levels of the compound, compound toxicities, and/or the progression of the disease, etc. In general, the dose equivalent of a compound is from about 1 μg/kg to 10 mg/kg for a typical subject. Many different administration methods are known to those of skill in the art.

For administration, compounds of the present invention can be administered at a rate determined by factors that can include, but are not limited to, the LD₅₀ of the compound, the pharmacokinetic profile of the compound, contraindicated drugs, and the side-effects of the compound at various concentrations, as applied to the mass and overall health of the subject. Administration can be accomplished via single or divided doses.

V. Methods for Treating or Preventing PI3K-Mediated Disorders and Conditions

The compounds of the present invention and pharmaceutical compositions comprising a compound of the present invention can be administered to a subject suffering from a PI3K-mediated disorder or condition. PI3K-mediated disorders and conditions can be treated prophylactically, acutely, and chronically using compounds of the present invention, depending on the nature of the disorder or condition. Typically, the host or subject in each of these methods is human, although other mammals can also benefit from the administration of a compound of the present invention.

In therapeutic applications, the compounds of the present invention can be prepared and administered in a wide variety of oral and parenteral dosage forms. Thus, the compounds of the present invention can be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally. In certain embodiments, the compounds of the present invention are delivered orally. The compounds can also be delivered rectally, bucally or by insufflation.

The compounds utilized in the pharmaceutical method of the invention can be administered at the initial dosage of about 0.001 mg/kg to about 100 mg/kg daily. In certain embodiments, the daily dose range is from about 0.1 mg/kg to about 10 mg/kg. The dosages, however, may be varied depending upon the requirements of the subject, the severity of the condition being treated, and the compound being employed. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The compounds of the invention can also be combined in a pharmacetical composition with compounds that are useful for the treatment of cancer (e.g., cytotoxic drugs such as TAXOL®, taxotere, GLEEVEC® (Imatinib Mesylate), adriamycin, daunomycin, cisplatin, etoposide, a vinca alkaloid, vinblastine, vincristine, methotrexate, or adriamycin, daunomycin, cis-platinum, etoposide, and alkaloids, such as vincristine, farnesyl transferase inhibitors, endostatin and angiostatin, VEGF inhibitors, and antimetabolites such as methotrexate. The compounds of the present invention may also be used in combination with a taxane derivative, a platinum coordination complex, a nucleoside analog, an anthracycline, a topoisomerase inhibitor, or an aromatase inhibitor).

The compounds of the invention can also be combined in a pharmacetical composition with compounds that are useful for the treatment of a thrombolytic disease, heart disease, stroke, etc., (e.g., aspirin, streptokinase, tissue plasminogen activator, urokinase, anticoagulants, antiplatelet drugs (e.g., PLAVIX®; clopidogrel bisulfate), a statin (e.g., LIPITOR® (Atorvastatin calcium), ZOCOR® (Simvastatin), CRESTOR® (Rosuvastatin), etc.), a Beta blocker (e.g, Atenolol), NORVASC® (amlodipine besylate), and an ACE inhibitor (e.g., lisinopril)).

The compounds of the invention can also be combined in a pharmacetical composition with compounds that are useful for the treatment of anti-hypertension agents such as, ACE inhibitors, lipid lowering agents such as statins, LIPITOR® (Atorvastatin calcium), calcium channel blockers such as NORVASC® (amlodipine besylate). The compounds of the present invention may also be used in combination with fibrates, beta-blockers, NEPI inhibitors, Angiotensin-2 receptor antagonists and platelet aggregation inhibitors.

For the treatment of inflammatory diseases, including rheumatoid arthritis, the compounds of the invention may be combined with agents such as TNF-α inhibitors such as anti-TNFα monoclonal antibodies (such as REMICADE®, CDP-870 and D2E7) and TNF receptor immunoglobulin molecules (such as ENBREL®), IL-1 inhibitors, receptor antagonists or soluble IL-1Rα (e.g. KINERET™ or ICE inhibitors), nonsteroidal anti-inflammatory agents (NSAIDS), piroxicam, diclofenac, naproxen, flurbiprofen, fenoprofen, ketoprofen ibuprofen, fenamates, mefenamic acid, indomethacin, sulindac, apazone, pyrazolones, phenylbutazone, aspirin, COX-2 inhibitors (such as CELEBREX® (celecoxib), VIOXX® (rofecoxib), BEXTRA® (valdecoxib and etoricoxib), metalloprotease inhibitors (preferably MMP-13 selective inhibitors), p2×7 inhibitors, a26 inhibitors, NEUROTIN®, pregabalin, low dose methotrexate, leflunomide, hydroxychloroquine, d-penicillamine, auranofin or parenteral or oral gold.

The compounds of the invention can also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib, valdecoxib, rofecoxib and etoricoxib, analgesics and intraarticular therapies such as corticosteroids and hyaluronic acids such as hyalgan and synvisc.

The compounds of the invention may also be used in combination with antiviral agents such as Viracept, AZT, aciclovir and famciclovir, and antisepsis compounds such as Valant.

The compounds of the present invention may also be used in combination with CNS agents such as antidepressants (such as sertraline), anti-Parkinsonian drugs (such as deprenyl, L-Dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase), and anti-Alzheimer's drugs such as donepezil, tacrine, a26 inhibitors, NEUROTIN®, pregabalin, COX-2 inhibitors, propentofylline or metryfonate.

The compounds of the present invention may also be used in combination with osteoporosis agents such as EVISTA® (raloxifene hydrochloride) droloxifene, lasofoxifene or fosomax and immunosuppressant agents such as FK-506 and rapamycin.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

Intermediate 1: 4-bromo-2-aminophenol. A mixture of 4-bromo-2-nitrophenol (25.0 g, 114.68 mmol) and Raney Nickel catalyst (9 g) in tetrahydrofuran (200 ml) was stirred at an initial H₂ psi of 69 for 1 hour. The Raney Nickel was filtered away and the reaction mixture was concentrated to give a dark brown solid. MS: M⁺+1=189 Da.

Intermediate 2: 4-bromo-N-(tert-butoxycarbonyl)-2-aminophenol. A mixture of 4-bromo-2-aminophenol (20.0 g, 106.4 mmol) and di-tert-butyldicarbonate (BOC)₂O (46.4 g, 212.7 mmol), in tetrahydrofuran (286 ml) was stirred at room temperature for 24 hours. After the tetrahydrofuran was removed under reduced pressure, the reaction mixture was diluted with methanol (50 ml), 1N sodium hydroxide (100 ml), and water (100 ml). The reaction mixture stirred for 30 minutes, the methanol was removed in vacuo and the basic reaction mixture was neutralized to a pH of about 7 using 1N hydrochloric acid. The product usually precipitates from solution as an oil and is dissolved in CH₂Cl₂, separated from the water layer, dried with magnesium sulfate and concentrated to give dark brown solid. MS: M⁺+1=187.9 Da.

Intermediate 3: 6-Bromo-2,3-dihydro-benzo[1,4]oxazine-4-carboxylic acid tert-butyl ester. A mixture of Intermediate 2 (20.0, 69.41 mmol), 1,2 dibromoethane (47.80 ml, 555.30 mmol), potassium carbonate (143.90 g, 1041.15 mmol) and benzyl triethylammonium chloride (7.90 g, 34.71 mmol) in 3-pentanone (700 ml) was stirred using a mechanical stirrer and heated to reflux for 18 hours. Upon cooling to room temperature, the potassium carbonate was filtered from the solution mixture and the 3-pentanone was removed in vacuo. The reaction mixture was diluted with ethyl acetate, washed with 0.5N sodium hydroxide, 0.1N HCl, and then brine. The organic layer was dried with magnesium sulfate and concentrated. MS: M⁺+1=315.2 Da.

Intermediate 4: 6-Formyl-2,3-dihydro-benzo[1,4]oxazine-4-carboxylic acid tert-butyl ester. To a −78° C. solution of ether (500 ml) and Intermediate 3 (5.00 g, 15.91 mmol) was added a 1.3 M solution of sec-BuLi in cyclohexane (36.72 ml, 47.74 mmol) dropwise. The reaction was stirred for 10 minutes and was quenched with DMF (12.32 ml, 159.10 mmol). The reaction was allowed to stir for an addition 10 minutes and was then quenched with acetic acid and warmed to room temperature. The ether was removed in vacuo and the reaction mixture was diluted with ethyl acetate, washed with 5% citric acid, NaHCO₃ and brine. The organic layer was dried with magnesium sulfate and concentrated. MS: M⁺−1=262.1 Da.

Intermediate 5: 3,4-Dihydro-2H-benzo[1,4]oxazine-6-carbaldehyde. To a 0° C. solution of dichloromethane (CH₂Cl₂)(80 ml) and Intermediate 4 (4.87 g, 18.50 mmol) was added, via an addition funnel, trifluoroacetic acid (25 ml). The reaction was stirred for 4 hours and warmed to room temperature. The CH₂Cl₂ was removed in vacuo and the reaction mixture was diluted with ethyl acetate, washed with NaHCO₃ and brine. The organic layer was dried with magnesium sulfate and concentrated to yield the title product. MS: M⁺+1=163.9 Da.

Intermediate 6: 4-(1-Phenyl-methanoyl-3,4-dihydro-2H-benzo[1,4]oxazine-6-carbaldehyde. To a solution of dichloromethane (6 ml) and Intermediate 5 (0.100 g, 0.613 mmol) was added triethylamine (0.128 ml, 0.919 mmol) followed by benzoyl chloride (0.0712 ml, 0.613 mmol). The reaction was then stirred at room temperature for 24 hours. The CH₂Cl₂ was removed in vacuo to yield the title product. MS: M⁺+1=268.1 Da.

### Example 1

5-[4-(1-Phenyl-methanoyl)-3,4-dihydro-2H-benzo[1,4]oxazine-6-ylmethylene]-2-thioxo-thiazolidin-4-one. To a solution of methanol (4 ml) and

Intermediate 6 (0.103 g, 0.385 mmol) was added ethylenediamine diacetate (0.069 g, 0.385 mmol) and rhodanine (0.051 g, 0.385 mmol). The reaction was stirred at room temperature overnight. The product precipitated from solution. The product was removed by filtration, washed with methanol and EtO₂ to give a yellow solid. MS: M⁺+1=383.0 Da.

Unless otherwise noted, the following Examples were synthesized in a manner analogous to Example 1.

### Example 2

5-[4-(1-Cyclohexyl-methanoyl)-3,4-dihydro-2H-benzo[1,4]oxazine-6-ylmethylene]-2-thioxo-thiazolidin-4-one. MS: M⁺+1=389.0 Da.

### Example 3

5-[(4-benzenesulfonyl)-3,4-dihydro-2H-benzo[1,4]oxazine-6-ylmethylene)-2-thioxo-thiazolidin-4-one. MS: M⁺−1=417.0 Da.

Intermediate 7: 4-(3,5-dimethyl-benzyl)-3,4-dihydro-2H-benzo[1,4]oxazine-6-carbaldehyde. To a solution of tetrahydrofuran (10 ml) and Intermediate 5 (0.150 g, 0.919 mmol) was added 2-tert-butylimino-2-diethylamino-1,3-dimethyl-perhydrol, 3,2-diazaphosphorine on polystyrene (BEMP resin) (0.877 g, 1.93 mmol) and 3,5-dimethylbenzyl bromide (0.220 g, 1.10 mmol). The reaction was stirred overnight. The BEMP resin was filtered away and the tetrahydrofuran was removed in vacuo. MS: M⁺−1=280.1 Da.

### Example 4

4-(3,5-dimethyl-benzyl)-3,4-dihydro-2H-benzo[1,4]oxazine-6-ylmethylene]-2-thioxo-thiazolidin-4-one. The title compound was synthesized in a manner analogous to Example 1 using Intermediate 7 (0.097 g, 0.345 mmol) and rhodanine (0.046 g, 0.345 mmol). MS: M⁺−1=264.2 Da.

Intermediate 8: 4-(3,5-Di-tert-butyl-benzyl)-3,4-dihydro-2H-benzo[1,4]oxazine-6-carbaldehyde. To a solution of DMF (14 ml) and Intermediate 5 (0.220 g, 1.35 mmol) was added sodium hydride (0.068 g, 2.84 mmol) and 3,5-di-tert-butyl-benzyl bromide (0.458 g, 1.62 mmol). The reaction was stirred at room temperature for 24 hours. The DMF was removed in vacuo and the reaction mixture was diluted with ethyl acetate, washed with 1N HCl, NaHCO₃, and then brine. The organic layer was dried with magnesium sulfate and concentrated. MS: M⁺−1=364.2 Da.

### Example 5

5[4-(3,5-Di-tert-butyl-benzyl)-3,4-dihydro-2H-benzo[1,4]oxazine-6-ylmethylene]-2-thioxo-thiazolidin-4-one. The title compound was synthesized in a manner analogous to Example 1. MS: M⁺−1=479.1 Da.

Intermediate 9: 2-Bromo-7,8-dihydro-6H-5-oxa-9-aza-benzocycloheptane-9-carboxylic acid tert-butyl ester. A mixture of Intermediate 2 (7.98 g, 27.69 mmol), 1,3 dibromopropane (22.48 ml, 221.56 mmol), and potassium carbonate (76.54 g, 553.8 mmol) in 3-pentanone (700 ml) was stirred using a mechanical stirrer and heated to reflux for 18 hours. Upon cooling to room temperature, the potassium carbonate was filtered from the solution mixture and the 3-pentanone was removed in vacuo. The reaction mixture was diluted with ethyl acetate, washed with 0.5N sodium hydroxide, 0.1N HCl, and then brine. The organic layer was dried with magnesium sulfate and concentrated. To the residue was added hexanes and ethyl ether and the precipitated solid was collected by filtration and dried to give the title compound. MS: M⁺−1=228.0 Da.

Intermediate 10: 2-Formyl-7,8-dihydro-6H-5-oxa-9-aza-benzocycloheptane-9-carboxylic acid tert-butyl ester. The title compound was synthesized in a manner analogous to Intermediate 4 using Intermediate 9 instead of Intermediate 3. MS: M⁺−1=276.1 Da.

Intermediate 11: 6,7,8,9-Tetrahydro-5-oxa-9-aza-benzocycloheptene-2-carbaldehyde. The title compound was synthesized in a manner analogous to Intermediate 5 using Intermediate 10 instead of Intermediate 4.

Intermediate 12: 9-(3,5-Dimethoxy-benzyl)-6,7,8,9-Tetrahydro-5-oxa-9-aza-benzocycloheptene-2-carbaldehyde. To a solution of dichloromethane (5.65 ml) and Intermediate 11 (0.100 g, 0.565 mmol) was added triethylamine (0.095 ml, 0.678 mmol) and 3,5-dimethoxybenzyl chloride (0.147 g, 0.734 mmol). The reaction was stirred for 24 hours at room temperature, and then an additional 24 hours at 30° C. The dichloromethane was removed in vacuo and the reaction mixture was diluted with ethyl acetate and washed with water. The organic layer was dried with magnesium sulfate and concentrated. MS: M⁺+1=328.2 Da.

### Example 6

5-[9-(3,5-Dimethoxy-benzyl)-6,7,8,9-tetrahydro-5-oxa-9-aza-benzocyclohepten-2-ylmethylene]-2-thioxo-thiazolidin-4-one. The title compound was synthesized in a manner analogous to Example 1 using Intermediate 12 instead of Intermediate 6. MS: M⁺−1=441.1 Da.

Intermediate 13: 9-(3,5-Dimethyl-benzyl)-6,7,8,9-Tetrahydro-5-oxa-9-aza-benzocycloheptene-2-carbaldehyde. The title compound was synthesized as in Intermediate 7 using Intermediate 11 (0.100 g, 0.565 mmol), BEMP resin (0.54 g, 1.187 mmol) and 3,5-dimethyl benzyl bromide (0.135 g, 0.678 mmol). MS: M⁺=296 Da.

### Example 7

5-[9-(3,5-Dimethyl-benzyl)-6,7,8,9-tetrahydro-5-oxa-9-aza-benzocyclohepten-2-ylmethylene]-2-thioxo-thiazolidin-4-one. The title compound was synthesized as Example 1 using Intermediate 13 (0.013 g, 0.044 mmol) and rhodanine (0.0059, 0.044 mmol). MS: M⁺−1=409.1 Da.

Intermediate 14: [5-Bromo-2-(2-bromo-propoxy)-phenyl]-carbamic acid tert-butyl ester. To a solution of dichloromethane (50 ml) and 2-bromo-propan-1-ol (0.482 g, 3,47 mmol) was added PPh₃ (triphenylphosphine) resin (2.16 g, 3.47 mmol), DEAD (diethyl azodicarboxylate) (0.594 g, 3.47 mmol) and Intermediate 2 (0.500 g, 1.74 mmol). The reaction was stirred for 24 hours at room temperature. The triphenyl phosphine resin was filtered from the reaction mixture and washed with dichloromethane and hexanes. The solvent was removed in vacuo and the reaction mixture was diluted with ethyl acetate, washed with 0.5 M sodium hydroxide and brine. The organic layer was dried with magnesium sulfate and concentrated. MS: M⁺+1=410.0 Da.

Intermediate 15: 6-Bromo-3-methyl-2,3-dihydro-benzo[1,4]oxazine-4-carboxylic acid. To a solution of 3-pentanone and Intermediate 14 (9.0 g, 22.05 mmol) was added potassium carbonate (45.71 g, 330.80 mmol) and benzyl triethylammonium chloride (2.51 g, 11.03 mmol). The reaction stirred using a mechanical stirrer and heated to reflux for 18 hours. Upon cooling to room temperature, the potassium carbonate was filtered from the solution mixture and the 3-pentanone was removed in vacuo. The reaction mixture was diluted with ethyl acetate, washed with 0.5N sodium hydroxide, 0.1N HCl and brine. The organic layer was dried with magnesium sulfate and concentrated. MS: M⁺−1=326.1 Da.

### Example 8

5-[3-Methyl-4-(-phenyl-methanoyl)-3,4-dihydro-2H-benzo[1,4]oxazine-6-ylmethylene]-2-thioxo-thiazolidin-4-one. The title product was synthesized in a manner analogous to Example 1, using Intermediate 15. MS: M⁺+1=395.0 Da.

### Example 9

5-[4-(3,5-Dimethyl-benzyl)-3-methyl-3,4-dihydro-2H-benzo[1,4]oxazine-6-ylmethylene]-2-thioxo-thiazolidin-4-one. The title compound was synthesized in a manner analogous to Example 8. MS: M⁺−1=409.1 Da.

### Example 10

5-(3,4-dihydro-2H-benzo[1,4]oxazine-6-ylmethylene)-2-thioxo-thiazolidin-4-one. Microanalysis (C₁₂H₁₀N₂O₂S₂): calculated: C=51.78; H=3.62; N=10.06; found: C=51.38; H=3.59; N=9.85. MS: M⁺−1=377.0 Da.

Intermediate 16: 4-(2-Naphthalen-2-yl-2oxo-ethyl)-3,4-dihydro-2H-benzo[1,4]oxazine-6-carbaldehyde. To a solution of 1,2 dichloroethane (3 ml) and Intermediate 5 (0.049 g, 0.300 mmol) was added triethylamine (0.054 ml, 0.390 mmol) followed by 2-naphthylacetyl chloride (0.068 g, 0.333 mmol). The reaction was stirred at room temperature for 24 hours. Isocyanate resin was added to scavenge excess Intermediate 5. The reaction was stirred for an additional 5 hours. Dichloromethane (2 ml) and saturated sodium bicarbonate (1 ml) were added to the reaction mixture and stirred for 10 minutes. The reaction mixture was filtered through a filter containing diatomaceous earth. The solvent was removed under reduced pressure to obtain the title product. MS: M⁺+1=332.1 Da.

### Example 11

5-[4-(2-Naphthalen-2-yl-acetyl)-3,4-dihydro-2H-benzo[1,4]oxazine-6-ylmethylene]-2-thioxo-thiazolidin-4-one The title compound was synthesized in a manner analogous to Example 1 from Intermediate 16. Microanalysis (C₂₄H₁₈N₂O₃S₂): calculated: C=64.55%; H=4.06%; N=6.27%; found: C=64.31%; H=3.42%; N=6.13%. MS: M⁺−1=445.0 Da.

### Example 12

5-[4-(Pyridine-4-carbonyl)]-3,4-dihydro-2H-benzo[1,4]oxazine-6-ylmethylene]-2-thioxo-thiazolidin-4-one. The title product was synthesized in a manner analogous to Example 11 using isonicotinoyl chloride instead of 2-naphthylacetyl chloride. In addition, the isocyanate resin was filtered from the reaction mixture and the mixture was washed with 5% citric acid, saturated sodium bicarbonate, brine, dried with magnesium sulfate, filtered and the organic layer was removed under reduced pressure to obtain 5-[4-(Pyridine-4-carbonyl)]-3,4-dihydro-2H-benzo[1,4]oxazine-6-carbaldehyde which was reacted in a manner analogous to that described in Example 1 to obtain the title product. Microanalysis (C₁₈H₁₃N₃O₃S₂): calculated: C=56.38%; H=3.42%; N=10.96%; O=12.52%; found: C=56.22%; H=3.06%; N=10.70%. MS: M⁺−1=382.9 Da.

### Example 13

5-[4-(Pyridine-3-carbonyl)]-3,4-dihydro-2H-benzo[1,4]oxazine-6-ylmethylene]-2-thioxo-thiazolidin-4-one. The title product was synthesized in a manner analogous to Example 12 using nicotinoyl chloride instead of isonicotinoyl chloride. Microanalysis (C₁₈H₁₃N₃O₃S₂): calculated: C=56.38%; H=3.42%; N=10.96%; found: C=56.01%; H=3.27%; N=11.02%. MS: M⁺−1=382.9 Da.

### Example 14

5-[4-(3,5-Dimethoxy-benzoyl)]-3,4-dihydro-2H-benzo[1,4]oxazine-6-ylmethylene]-2-thioxo-thiazolidin-4-one. The title product was synthesized in a manner analogous to Example 12, using dimethoxybenzoyl chloride instead of 2-naphthylacetyl chloride. Microanalysis (C₂₁H₁₈N₂O₅S₂): calculated: C=57.00%; H=4.10%; N=6.33%; found: C=56.56%; H=4.65%; N=6.49%. MS: M⁺−1=441.0 Da.

Intermediate 17: 4-[2-(3,4-Dichloro-phenyl)-acetyl]-3,4-dihydro-2H-benzo[1,4]oxazine-6-carbaldehyde. To a solution of 1,2 dichloroethane (10 ml) and Intermediate 5 (0.300 g, 1.84 mmol) was added triethylamine (0.333 ml, 2.39 mmol) followed by 3,4-dichlorophenyl acetyl chloride (0.452 g, 2.39 mmol). The reaction was stirred at room temperature for 24 hours. The 1,2-dichloroethane was removed under reduced pressure. The crude material was diluted with ethyl acteate, washed with 5% citric acid, saturated sodium bicarbonate, brine, dried with magnesium sulfate, filtered and the organic layer was removed under reduced pressure to obtain the title product.

### Example 15

4-[2-(3,4-Dichloro-phenyl)-acetyl]-3,4-dihydro-2H-benzo 1,4]oxazine-6-ylmethylene]-2-thioxo-thiazolidin-4-one. The title compound was synthesized in a manner analogous to Example 1 from Intermediate 17, with the exception that the filtered product was dissolved in hot DMF and recrystallized with a minimal amount of methanol. Microanalysis (C₂₀H₁₄Cl₂N₂O₃S₂): calculated: C=51.62%; H=3.03%; N=6.02%; found: C=51.48%; H=2.67%; N=5.82%. MS: M⁺+1=464.9 Da.

Intermediate 18: 9-Phenethyl-6,7,8,9-Tetrahydro-5-oxa-9-aza-benzocycloheptene-2-carbaldehyde. To a vial containing sodium hydride (0.045 g, 1.86 mmol) was added DMF (10 ml) followed by Intermediate 11. The reaction stirred for 15 minutes at room temperature and then 2-(bromo-ethyl)benzene (0.375 g, 1.36 mmol) was added to the reaction. Then another equivalent of sodium hydride (0.045 g, 1.86 mmol) was added followed by an equivalent of potassium iodide and the reaction was heated to 50° C. The temperature was increased by 20° C. every two hours until it reached a maximum of 130° C. The DMF was removed under reduced pressure. The remaining crude material was diluted in ethyl acetate, washed with 5% citric acid, saturated sodium bicarbonate, brine, dried with magnesium sulfate, filtered and removed organic layer under reduced pressure to obtain the title product.

### Example 16

5-(9-Phenethyl-6,7,8,9-tetrahydro-5-oxa-9-aza-benzocyclohepten-2-ylmethylene)-2-thioxo-thiazolidin-4-one. The title compound was synthesized in a manner analogous to Example 1 using

Intermediate 18. Microanalysis (C₂₁H₂₀N₂O₂S₂): calculated: C=63.61%; H=5.08%; N=7.06%; found: C=63.75%; H=4.51%; N=7.01%. MS: M⁺−1=395.0 Da.

Examples 17-72 and 104-127 were synthesized in the following fashion using Intermediate 5 or Intermediate 11.

The desired acid chlorides (e.g., R⁶-L-C(O)—Cl), isocyanates (e.g., R⁶-L-N═C═O) or chloroformates (e.g., R⁶-L-O—C(O)—Cl) (0.33 mmol of each) and 1,2 dichloroethane (1.5 ml) were placed into the appropriate reaction vessels. A stock solution (1.5 ml) that is 0.194 M for Intermediate 5 or 11 and 0.258 M in triethyl amine in a solution of 1,2 dichlorethane was delivered to each of the respective reaction vessels. The closed vessel was allowed to agitate for 24 hours, treated with an excess of Argonaut PS Isocyanate resin and agitation continued for an additional 24 hour period. The reactions where treated with NaHCO₃ (1.0 ml, saturated water solution) and 1,2 dichloroethane (2 ml), filtered through a pad of diatomaceous earth into tared vessels and the solvent removed under reduced pressure. The reactions where diluted with methanol to obtain a final molarity of approximately 0.2 M. An appropriate volume of a stock solution (0.2 M rhodanine and 0.2 M in ethylenediamine) was delivered to each reaction vessel. The reaction where allowed to agitate for 24 hours and the product recovered by filtration. The filtered products were dissolved in hot DMF and recrystallized in methanol to afford the desired title compounds.

### Example 17

6-(4-Oxo-2-thioxo-thiazolidin-5-ylidenemethyl)-2,3-dihydro-1,4-benzoxazine-4-carboxylic acid phenyl ester. MS: M⁺−1=397.9 Da.

### Example 18

6-(4-Oxo-2-thioxo-thiazolidin-5-ylidenemethyl)-2,3-dihydro-1,4-benzoxazine-4-carboxylic acid p-tolyl ester. MS: M⁺−1=41 1 Da.

### Example 19

5-(4-Phenylacetyl-3,4-dihydro-2H-1,4-benzoxazin-6-ylmethylene)-2-thioxo-thiazolidin-4-one. MS: M⁺−1=396 Da.

### Example 20

5-{4-[2-(3,4-Dimethoxy-phenyl)-acetyl]-3,4-dihydro-2H-1,4-benzoxazin-6-ylmethylene}-2-thioxo-thiazolidin-4-one. MS: M⁺−1=455 Da.

### Example 21

5-[4-(3-Methoxy-benzoyl)-3,4-dihydro-2H-1,4-benzoxazin-6-ylmethylene]-2-thioxo-thiazolidin-4-one. MS: M⁺−1=411 Da.

### Example 22

2-Thioxo-5-[4-(3,4,5-trimethoxy-benzoyl)-3,4-dihydro-2H-1,4-benzoxazin-6-ylmethylene]-thiazolidin-4-one. MS: M⁺−1=471 Da.

### Example 23

5-[4-(3-Methyl-benzoyl)-3,4-dihydro-2H-1,4-benzoxazin-6-ylmethylene]-2-thioxo-thiazolidin-4-one. MS: M⁺−1=395 Da.

### Example 24

5-[4-(Biphenyl-4-carbonyl)-3,4-dihydro-2H-1,4-benzoxazin-6-ylmethylene]-2-thioxo-thiazolidin-4-one. MS: M⁺−1=457.1 Da.

### Example 25

5-[4-(4-tert-Butyl-benzoyl)-3,4-dihydro-2H-1,4-benzoxazin-6-ylmethylene]-2-thioxo-thiazolidin-4-one. MS: M⁺−1=437.2 Da.

### Example 26

5-[4-(4-Ethyl-benzoyl)-3,4-dihydro-2H-1,4-benzoxazin-6-ylmethylene]-2-thioxo-thiazolidin-4-one. MS: M⁺−1=409.2 Da.

### Example 27

5-[4-(4-Hexyl-benzoyl)-3,4-dihydro-2H-1,4-benzoxazin-6-ylmethylene]-2-thioxo-thiazolidin-4-one. MS: M⁺−1=465.2 Da.

### Example 28

4-[6-(4-Oxo-2-thioxo-thiazolidin-5-ylidenemethyl)-2,3-dihydro-1,4-benzoxazine-4-carbonyl]-benzonitrile. MS: M⁺−1=406.1 Da.

### Example 29

5-[4-(Naphthalene-2-carbonyl)-3,4-dihydro-2H-1,4-benzoxazin-6-ylmethylene]-2-thioxo-thiazolidin-4-one. MS: M⁺−1=431.1 Da.

### Example 30

5-[4-(2-Phenyl-butyryl)-3,4-dihydro-2H-1,4-benzoxazin-6-ylmethylene]-2-thioxo-thiazolidin-4-one. MS: M⁺−1=423 Da.

### Example 31

5-(4-Isobutyryl-3,4-dihydro-2H-1,4-benzoxazin-6-ylmethylene)-2-thioxo-thiazolidin-4-one. MS: M⁺−1=347 Da.

### Example 32

5-(4-Cyclopropanecarbonyl-3,4-dihydro-2H-1,4-benzoxazin-6-ylmethylene)-2-thioxo-thiazolidin-4-one. MS: M⁺−1=345 Da.

### Example 33

5-(4-Cyclopentanecarbonyl-3,4-dihydro-2H-1,4-benzoxazin-6-ylmethylene)-2-thioxo-thiazolidin-4-one. MS: M⁺−1=373 Da.

### Example 34

5-(4-Heptanoyl-3,4-dihydro-2H-1,4-benzoxazin-6-ylmethylene)-2-thioxo-thiazolidin-4-one. MS: M⁺−1=389 Da.

### Example 35

5-[4-(2-Thiophen-2-yl-acetyl)-3,4-dihydro-2H-1,4-benzoxazin-6-ylmethylene]-2-thioxo-thiazolidin-4-one. MS: M⁺−1=401.9 Da.

### Example 36

5-[4-(3-Cyclopentyl-propionyl)-3,4-dihydro-2H-1,4-benzoxazin-6-ylmethylene]-2-thioxo-thiazolidin-4-one. MS: M⁺−1=401.1 Da.

### Example 37

5-[4-(3-Phenyl-acryloyl)-3,4-dihydro-2H-1,4-benzoxazin-6-ylmethylene]-2-thioxo-thiazolidin-4-one. MS: M⁺−1=406.9 Da.

### Example 38

5-[4-(2-Phenoxy-acetyl)-3,4-dihydro-2H-1,4-benzoxazin-6-ylmethylene]-2-thioxo-thiazolidin-4-one. MS: M⁺−1=411 Da.

### Example 39

5-[4-(2-Benzyloxy-acetyl)-3,4-dihydro-2H-1,4-benzoxazin-6-ylmethylene]-2-thioxo-thiazolidin-4-one. MS: M⁺−1=425 Da.

### Example 40

5-[4-(2-Phenylsulfanyl-acetyl)-3,4-dihydro-2H-1,4-benzoxazin-6-ylmethylene]-2-thioxo-thiazolidin-4-one. MS: M⁺−1=427 Da.

### Example 41

5-[4-(Furan-2-carbonyl)-3,4-dihydro-2H-1,4-benzoxazin-6-ylmethylene]-2-thioxo-thiazolidin-4-one. MS: M⁺−1=471.9 Da.

### Example 42

5-[4-(Thiophene-2-carbonyl)-3,4-dihydro-2H-1,4-benzoxazin-6-ylmethylene]-2-thioxo-thiazolidin-4-one. MS: M⁺−1=387.9 Da.

### Example 43

5-[4-(Quinoxaline-2-carbonyl)-3,4-dihydro-2H-1,4-benzoxazin-6-ylmethylene]-2-thioxo-thiazolidin-4-one. MS: M⁺−1=433 Da.

### Example 44

8-Oxo-8-[6-(4-oxo-2-thioxo-thiazolidin-5-ylidenemethyl)-2,3-dihydro-1,4-benzoxazin-4-yl]-octanoic acid methyl ester. MS: M⁺−1=447.1 Da.

### Example 45

5-[4-(3,5-Bis-trifluoromethyl-benzoyl)-3,4-dihydro-2H-1,4-benzoxazin-6-ylmethylene]-2-thioxo-thiazolidin-4-one. MS: M⁺−1=517.9 Da.

### Example 46

5-[4-(3,5-Difluoro-benzoyl)-3,4-dihydro-2H-1,4-benzoxazin-6-ylmethylene]-2-thioxo-thiazolidin-4-one. MS: M⁺−1=417.9 Da.

### Example 47

5-{4-[2-(4-Chloro-phenoxy)-acetyl]-3,4-dihydro-2H-1,4-benzoxazin-6-ylmethylene}-2-thioxo-thiazolidin-4-one. MS: M⁺−1=445 Da.

### Example 48

5-[4-(2,2-Difluoro-1,3-benzodioxole-5-carbonyl)-3,4-dihydro-2H-1,4-benzoxazin-6-ylmethylene]-2-thioxo-thiazolidin-4-one. MS: M⁺−1=461.9 Da.

### Example 49

5-[4-(Isoxazole-5-carbonyl)-3,4-dihydro-2H-1,4-benzoxazin-6-ylmethylene]-2-thioxo-thiazolidin-4-one. MS: M⁺−1=372.9 Da.

### Example 50

6-(4-Oxo-2-thioxo-thiazolidin-5-ylidenemethyl)-2,3-dihydro-1,4-benzoxazine-4-carboxylic acid 4-methoxycarbonyl-phenyl ester. MS: M⁺−1=455 Da.

### Example 51

5-[4-(2,5-Dichloro-thiophene-3-carbonyl)-3,4-dihydro-2H-1,4-benzoxazin-6-ylmethylene]-2-thioxo-thiazolidin-4-one. MS: M⁺−1=456.9 Da.

### Example 52

5-[4-(5-Methyl-isoxazole-3-carbonyl)-3,4-dihydro-2H-1,4-benzoxazin-6-ylmethylene]-2-thioxo-thiazolidin-4-one. MS: M⁺−1=386.9 Da.

### Example 53

5-[4-(3-Chloro-thiophene-2-carbonyl)-3,4-dihydro-2H-1,4-benzoxazin-6-ylmethylene]-2-thioxo-thiazolidin-4-one. MS: M⁺−1=421.9 Da.

### Example 54

5-[4-(Pyridine-2-carbonyl)-3,4-dihydro-2H-1,4-benzoxazin-6-ylmethylene]-2-thioxo-thiazolidin-4-one. MS: M⁺−1=382.9 Da.

### Example 55

6-(4-Oxo-2-thioxo-thiazolidin-5-ylidenemethyl)-2,3-dihydro-1,4-benzoxazine-4-carboxylic acid (3-trifluoromethyl-phenyl)-amide. MS: M⁺−1=464.1 Da.

### Example 56

6-(4-Oxo-2-thioxo-thiazolidin-5-ylidenemethyl)-2,3-dihydro-1,4-benzoxazine-4-carboxylic acid (2-trifluoromethoxy-phenyl)-amide. MS: M⁺−1=480.1 Da.

### Example 57

6-(4-Oxo-2-thioxo-thiazolidin-5-ylidenemethyl)-2,3-dihydro-1,4-benzoxazine-4-carboxylic acid p-tolylamide. MS: M⁺−1=410.1 Da.

### Example 58

6-(4-Oxo-2-thioxo-thiazolidin-5-ylidenemethyl)-2,3-dihydro-1,4-benzoxazine-4-carboxylic acid phenylamide. MS: M⁺−1=396.1 Da.

### Example 59

6-(4-Oxo-2-thioxo-thiazolidin-5-ylidenemethyl)-2,3-dihydro-1,4-benzoxazine-4-carboxylic acid (3-methoxy-phenyl)-amide. MS: M⁺−1=426.1 Da.

### Example 60

5-[4-(7,7-Dimethyl-2-oxo-bicyclo[2.2.1]hept-1-ylmethanesulfonyl)-3,4-dihydro-2H-1,4-benzoxazin-6-ylmethylene]-2-thioxo-thiazolidin-4-one. MS: M⁺−1=419.2 Da.

### Example 61

5-[4-(Benzofurazan-5-carbonyl)-3,4-dihydro-2H-1,4-benzoxazin-6-ylmethylene]-2-thioxo-thiazolidin-4-one. MS: M⁺−1=423.9 Da.

### Example 62

6-(4-Oxo-2-thioxo-thiazolidin-5-ylidenemethyl)-2,3-dihydro-1,4-benzoxazine-4-carboxylic acid octylamide. MS: M⁺−1=432 Da.

### Example 63

5-[4-(3,5-Dichloro-benzoyl)-3,4-dihydro-2H-1,4-benzoxazin-6-ylmethylene]-2-thioxo-thiazolidin-4-one. MS: M⁺−1=449.9 Da.

### Example 64

6-(4-Oxo-2-thioxo-thiazolidin-5-ylidenemethyl)-2,3-!dihydro-1,4-benzoxazine-4-carboxylic acid (2-thiophen-2-yl-ethyl)-amide. MS: M-1=429.9 Da.

### Example 65

6-(4-Oxo-2-thioxo-thiazolidin-5-ylidenemethyl)-2,3-dihydro-1,4-benzoxazine-4-carboxylic acid phenethyl-amide. MS: M⁺−1=424 Da.

### Example 66

6-(4-Oxo-2-thioxo-thiazolidin-5-ylidenemethyl)-2,3-dihydro-1,4-benzoxazine-4-carboxylic acid (4-phenoxy-phenyl)-amide. MS: M⁺−1=488 Da.

### Example 67

6-(4-Oxo-2-thioxo-thiazolidin-5-ylidenemethyl)-2,3-dihydro-1,4-benzoxazine-4-carboxylic acid (3,5-dimethoxy-phenyl)-amide. MS: M⁺−1=456 Da.

### Example 68

6-(4-Oxo-2-thioxo-thiazolidin-5-ylidenemethyl)-2,3-dihydro-1,4-benzoxazine-4-carboxylic acid cyclopentylamide. MS: M⁺−1=388 Da.

### Example 69

6-(4-Oxo-2-thioxo-thiazolidin-5-ylidenemethyl)-2,3-dihydro-1,4-benzoxazine-4-carboxylic acid naphthalen-1-yl ester. MS: M⁺−1=447 Da.

### Example 70

5-[4-(3-Phenyl-propionyl)-3,4-dihydro-2H-1,4-benzoxazin-6-ylmethylene]-2-thioxo-thiazolidin-4-one. MS: M⁺−1=409 Da.

### Example 71

5-[4-(1,3-Benzodioxole-5-carbonyl)-3,4-dihydro-2H-1,4-benzoxazin-6-ylmethylene]-2-thioxo-thiazolidin-4-one. MS: M⁺−1=425 Da.

### Example 72

5-[4-(4-Methanesulfonyl-benzoyl)-3,4-dihydro-2H-benzol, 4]oxazine-6-ylmethylene]-2-thioxo-thiazolidin-4-one. MS: M⁻=459.0 Da.

### Example 73

4-[2-(3,5-Dimethoxy-phenyl)-acetyl]3,4-dihydro-2H-benzol, 4]oxazine-6-ylmethylene]-2-thioxo-thiazolidin-4-one. Microanalysis (C₂₂H₂₀N₂O₅S₂): calculated: C=57.88%; H=4.42%; N=6.14%; O=17.52%; S=14.05%; observed: C=57.24%; H=4.52%; N=6.30%. MS: M⁻=455.0 Da.

### Example 74

4-[4-(4-Methyl-piperazin-1-ylmethyl)-benzoyl]-3,4-dihydro-2H-benzol, 4]oxazine-6-ylmethylene]-2-thioxo-thiazolidin-4-one. Microanalysis (C₂₅H₂₆N₄O₃S₂): calculated: C=60.71%; H=4.30%; N=11.33%; O=9.70%; S=12.96%; observed: C=59.20%; H=5.12%; N=10.58%. MS: M⁻=493.3 Da.

### Example 75

6-(4-Oxo-2-thioxo-thiazolidin-5-ylidenemethyl)-2,3-dihydro-benzo[14]oxazine-4-carboxylic acid (3,4-dimethoxy-phenyl)-aniide. The title product was synthesized in a manner analogous to Example 1 using 4-isocyanato-1,2-dimethoxy-benzene. Microanalysis (C₂₁H₁₉N₃O₅S₂): calculated: C=55.13%; H=4.19%; N=9.18%; O=17.48%; S=14.02%; observed: C=52.35%; H=3.32%; N=8.65%. MS: M⁺/M⁻=458.1/456.1 Da.

### Example 76

6-(4-Oxo-2-thioxo-thiazolidin-5-ylidenemethyl)-2,3-dihydro-benzo[14]oxazine-4-carboxylic acid (3,5-dichloro-phenyl)-amide. The title product was synthesized in a manner analogous to Example 1 using 1,3-dichloro-5-isocyanato-benzene. Microanalysis (C₁₉H₁₃Cl₂N₃O₃S₂): calculated: C=48.93%; H=2.81%; N=9.01%; O=10.29%; S=13.75%; observed: C=46.58%; H=1.98%; N=8.22%. MS: M⁺/M⁻=465.9/464.9 Da.

### Example 77

6-(4-Oxo-2-thioxo-thiazolidin-5-ylidenemethyl)-2,3-dihydro-benzo[1,4]oxazine-4-carboxylic acid (4-chloro-phenyl)-amide. The title product was synthesized in a manner analogous to Example 1 using 1-chloro-4-isocyanato-benzene. Microanalysis (C₁₉H₁₄ClN₃O₃S₂): calculated: C=52.84%; H=3.27%; N=9.73%; O=11.11%; S=14.85%; observed: C=52.65%; H=2.77%; N=5.93%. MS: M⁺/M⁻=433.0/431.0 Da.

### Example 78

6-(4-Oxo-2-thioxo-thiazolidin-5-ylidenemethyl)-2,3-dihydro-benzo[1,4]oxazine-4-carboxylic acid (3,4-dichloro-phenyl)-amide. The title product was synthesized in a manner analogous to Example 1 using 1,2-dichloro-4-isocyanato-benzene Microanalysis (Cl ₉H₃Cl₂N₃O₃S₂): calculated: C=48.93%; H=2.81%; N=9.01%; O=10.29%; S=13.75%; observed: C=46.97%; H=2.89%; N=8.31%. MS: M⁺/M⁻=467.9/464.9 Da.

### Example 79

6-(4-Oxo-2-thioxo-thiazolidin-5-ylidenemethyl)-2,3-dihydro-benzo[14]oxazine-4-carboxylic acid (3,5-dimethyl-phenyl)-amide. The title product was synthesized in a manner analogous to Example 1 using 1,3-dimethyl-5-isocyanato-benzene. Microanalysis (C₂₁H₁₉N₃O₃S₂): calculated: C=59.28%; H=4.50%; N=9.87%; O=11.28%; S=15.07%; observed: C=58.48%; H=4.32%; N=9.65%. MS: M⁺/M⁻=426.1/424.1 Da.

### Example 80

6-(4-Oxo-2-thioxo-thiazolidin-5-ylidenemethyl)-2,3-dihydro-benzo[1,4]oxazine-4-carboxylic acid (3-chloro-phenyl)-amide. The title product was synthesized in a manner analogous to Example 1 using 1-chloro-3-isocyanato-benzene. Microanalysis (C₁₉H₁₄ClN₃O₃S₂): calculated: C=52.84%; H=3.27%; N=9.73%; O=11.11%; S=14.85%; observed: C=52.82%; H=2.96%; N=9.72%. MS: M⁺/M⁻=432.0/430.0 Da.

### Example 81

5-[4-(3,5-Di-tert-butyl-benzoyl)-3,4-dihydro-2H-benzo[1,4]oxazin-6-ethylene]-2-thioxo-thiazolidin-4-one. Microanalysis (C₂₇H₃₀N₂O₃S₂): calculated: C=65.56%; H=6.11%; N=5.66%; O=9.70%; S=12.96%; observed: C=62.78%; H=5.97%; N=5.22%. MS: M+/M⁻=495.1/493.1 Da.

### Example 82

5-[4-(4-Phenyl-butryl)-3,4-dihydro-2H-benzo[1,4]oxazin-6-ethylene]-2-thioxo-thiazolidin-4-one. Microanalysis (C₂₂H₂₀N₂O₃S₂): calculated: C=62.24%; H=4.75%; N=6.60%; O=11.31%; S=15.11%; observed: C=62.00%; H=4.27%; N=6.49%. MS: M⁺/M⁻=425.1/423.1 Da.

### Example 83

5-[4-Cycloheptanecarbonyl-3,4-dihydro-2H-benzo[1,4]oxazin-6-ethylene]-2-thioxo-thiazolidin-4-one. Microanalysis (C₂₀H₂₂N₂O₃S₂): calculated: C=59.68%; H=5.51%; N=6.96%; O=11.92%; S=15.93%; observed: C=59.72%; H=5.16%; N=6.86%. MS: M⁺/M⁻=403.1/401.1 Da.

### Example 84

5-[4-(2-Phenyl-propionyl)-3,4-dihydro-2H-benzo[1,4]oxazin-6-ethylene]-2-thioxo-thiazolidin-4-one. Microanalysis (C₂₁H₁₈N₂O₃S₂): calculated: C=61.44%; H=4.42%; N=6.82%; O=11.69%; S=15.62%; observed: C=61.09%; H=4.18%; N=6.75%. MS: M⁺/M⁻=411.1/410.1 Da.

### Example 85

5-[4-(3-Methyl-cyclohexanecarbonyl)-3,4-dihydro-2H-benzo[1,4]oxazin-6-ethylene]-2-thioxo-thiazolidin-4-one. Microanalysis (C₂₀H₂₂N₂O₃S₂): calculated: C=59.68%; H=5.51%; N=6.96%; O=11.92%; S=15.93%; observed: C=59.53%; H=4.96%; N=6.25%. MS: M⁺/M⁻=403.1/401.1 Da.

### Example 86

5-[4-(2,3-Dimethyl-butyryl)-3,4-dihydro-2H-benzo[1,4]oxazin-6-ethylene]-2-thioxo-thiazolidin-4-one. Microanalysis (C₁₈H₂₀N₂O₃S₂): calculated: C=57.42%; H=5.35%; N=7.44%; O=12.75%; S=17.03%; observed: C=57.14%; H=5.07%; N=7.20%. MS: M⁺/M⁻=377.0/375.0 Da.

### Example 87

5-[4-(2-Methyl-cyclohexyl)-propionyl]-3,4-dihydro-2H-benzo[1,4]oxazin-6-ethylene]-2-thioxo-thiazolidin-4-one. Microanalysis (C₂₂H₂₆N₂O₃S₂): calculated: C=61.37%; H=6.09%; N=6.51%; O=11.15%; S=14.89%; observed: C=60.78%; H=6.13%; N=6.41%. MS: M⁺/M⁻=431.1/429.1 Da.

### Example 88

5-[4-(2-Methoxy-5-methyl-benzoyl)-3,4-dihydro-2H-benzo[1,4]oxazin-6-ethylene]-2-thioxo-thiazolidin-4-one. Microanalysis (C₂₁H₁₈N₂O₄S₂): calculated: C=59.14%; H=4.25%; N=6.57%; O=15.00%; S=15.04%; observed: C=59.04%; H=3.99%; N=6.34%. MS: M⁺/M⁻=427.1/425.1 Da.

### Example 89

5-[4-(2-Fluoro-5-methyl-benzoyl)-3,4-dihydro-2H-benzo[1,4]oxazin-6-ethylene]-2-thioxo-thiazolidin-4-one. Microanalysis (C₂₀H₁₅FN₂O₃S₂): calculated: C=57.96%; H=3.65%; N=6.76%; O=11.58%; S=15.47%; observed: C=58.10%; H=3.08%; N=5.80%. MS: M⁺/M⁻=415.0/413.0 Da.

### Example 90

2-Thioxo-5-[4-(2,3,3-trimethyl-butyryl)-3,4-dihydro-2H-benzo[1,4]oxazin-6-ethylene]-2-thioxo-thiazolidin-4-one. Microanalysis (C₁₉H₂₂N₂O₃S₂): calculated: C=58.44%; H=5.68%; N=7.17%; O=12.29%; S=16.42%; observed: C=58.36%; H=5.27%; N=7.01%. MS: M⁺/M⁻=391.1/389.1 Da.

### Example 91

5-[4-(2-Methyl-cyclohexanecarbonyl)-3,4-dihydro-2H-benzo[1,4]oxazin-6-ethylene]-2-thioxo-thiazolidin-4-one. Microanalysis (C₂₀H₂₂N₂O₃S₂): calculated: C=59.68%; H=5.51%; N=6.96%; O=11.92%; S=15.93%; observed: C=59.31%; H=5.29%; N=6.81%. MS: M⁺/M⁻=403.0/401.0 Da.

### Example 92

5-(4-Acetyl-3,4-dihydro-2H-1,4-benzoxazin-6-ylmethylene)-2-thioxo-thiazolidin-4-one. MS: M⁻=320 Da. Microanalysis (C₁₄H₁₂N₂O₃S₂): calculated: C=52.52%; H, 3.77%; N=8.74%; observed: C=52.49%; H=3.55%; N=8.59%.

### Example 93

4-(4-Propionyl-3,4-dihydro-2H-1,4-benzoxazin-6-ylmethylene)-2-thioxo-thiazolidin-4-one. MS: M⁻=334 Da. Microanalysis (C₁₅H₁₄N₂O₃S₂.0.90H₂O): calculated: C=51.42%; H=4.54%; N=7.99%; observed: C=51.39%; H=4.57%; N=7.99%.

### Example 94

5-(4-Butyryl-3,4-dihydro-2H-1,4-benzoxazin-6-ylmethylene)-2-thioxo-thiazolidin-4-one. MS: M⁻=348 Da. Microanalysis (C₁₆H₁₆N₂O₃S₂.0.20H₂O): calculated: C=54.60%; H=4.75%; N=7.95%; observed: C=54.66%; H=4.68%; N=7.93%.

### Example 95

5-(4-Hexanoyl-3,4-dihydro-2H-1,4-benzoxazin-6-ylmethylene)-2-thioxo-thiazolidin-4-one. MS: M⁻=375 Da. Microanalysis (C₁₈H₂₀N₂O₃S₂): calculated: C=57.46%; H=5.35%; N=7.44%; observed: C=57.07%; H=5.26%; N=7.32%.

### Example 96

5-(4-Pentanoyl-3,4-dihydro-2H-1,4-benzoxazin-6-ylmethylene)-2-thioxo-thiazolidin-4-one. MS: M⁻=362 Da. Microanalysis (C₁₇H₁₈N₂O₃S₂): calculated: C=56.37%; H=5.00%; N=7.73%; observed: C=56.00%; H=4.69%; N=7.73%.

### Example 97

5-[4-(2,2-Dimethyl-propionyl)-3,4-dihydro-2H-1,4-benzoxazin-6-ylmethylene]-2-thioxo-thiazolidin-4-one. MS: M⁻=361 Da. Microanalysis (C₁₇H₁₈N₂O₃S₂): calculated: C=56.33%; H=5.01%; N=7.73%; observed: C=56.05%; H=4.71%; N=7.55%.

### Example 98

5-[4-(4-Nonanoyl)-3,4-dihydro-2H-1,4-benzoxazin-6-ylmethylene]-2-thioxo-thiazolidin-4-one. MS: M⁻=417 Da. Microanalysis (C₂₁H₂₆N₂O₃S₂): calculated: C=60.40%; H=6.03%; N=6.71%; observed: C=60.23%; H=6.29%; N=6.66%.

### Example 99

5-[4-(2-Ethyl-hexanoyl)-3,4-dihydro-2H-1,4-benzoxazin-6-ylmethylene]-2-thioxo-thiazolidin-4-one. MS: M⁻=404 Da. Microanalysis (C₂₀GH₂₄N₂O₃S₂): calculated: C=59.38%; H=5.98%; N=6.92%; observed: C=59.36%; H=5.86%; N=6.88%.

### Example 100

Acetic acid 1-methyl-2-oxo-2-[6-(4-oxo-2-thioxo-thiazolidin-5-ylidenemethyl)-2,3-dihydro-1,4-benzoxazin-4-yl]-ethyl ester. MS: M⁻=391 Da. Microanalysis (C₁₇H₁₆N₂O₅S₂): calculated: C=52.19%; H, 3.83%; N=7.15%; observed: C=52.24%; H, 3.90%; N=6.82%.

### Example 101

4-Oxo-4-[6-(4-oxo-2-thioxo-thiazolidin-5-ylidenemethyl)-2,3-dihydro-1,4-benzoxazin-4-yl]-butyric acid methyl ester. MS: M⁻=391 Da. Microanalysis (C₁₇H₁₆N₂O₅S₂.0.2 mol H₂O): calculated: C=51.73%; H, 3.90%; N=7.09%; observed: C=51.56%; H, 3.87%; N=6.81%.

### Example 102

5-[4-(1-Acetyl-piperidine-4-carbonyl)-3,4-dihydro-2H-1,4-benzoxazin-6-ylmethylene]-2-thioxo-thiazolidin-4-one. MS: M⁻=431 Da. Microanalysis (C₂₀H₂₁N₃O₄S₂.0.2 mol H₂O): calculated: C=55.25%; H=4.92%; N=9.66%; observed: C=55.17%; H=4.64%; N=9.80%.

### Example 103

5-(6,7,8,9-Tetrahydro-5-oxa-9-aza-benzocyclohepten-2-ylmethylene)-2-thioxo-thiazolidin-4-one. The title product was synthesized in a manner analogous to Example 6 using Intermediate 11. MS: M⁻=292 Da.

### Example 104

5-(9-Cyclopropanecarbonyl-6,7,8,9-tetrahydro-5-oxa-9-aza-benzocyclohepten-2-ylmethylene)-2-thioxo-thiazolidin-4-one. MS: M⁺−1=346 Da.

### Example 105

2-(4-Oxo-2-thioxo-thiazolidin-5-ylidenemethyl)-7,8-dihydro-6H-5-oxa-9-aza-benzocycloheptene-9-carboxylic acid phenyl ester. MS: M⁺−1=398 Da.

### Example 106

2-(4-Oxo-2-thioxo-thiazolidin-5-ylidenemethyl)-7,8-dihydro-6H-5-oxa-9-aza-benzocycloheptene-9-carboxylic acid naphthalen-1-yl ester. MS: M⁺−1=462 Da.

### Example 107

2-(4-Oxo-2-thioxo-thiazolidin-5-ylidenemethyl)-7,8-dihydro-6H-5-oxa-9-aza-benzocycloheptene-9-carboxylic acid p-tolyl ester. MS: M⁺−1=412 Da.

### Example 108

2-(4-Oxo-2-thioxo-thiazolidin-5-ylidenemethyl)-7,8-dihydro-6H-5-oxa-9-aza-benzocycloheptene-9-carboxylic acid 4-methoxycarbonyl-phenyl ester. MS: M⁺−1=470 Da.

### Example 109

5-{9-[2-(4-Chloro-phenoxy)-acetyl]-6,7,8,9-tetrahydro-5-oxa-9-aza-benzocyclohepten-2-ylmethylene}-2-thioxo-thiazolidin-4-one. MS: M⁺−1=461 Da.

### Example 110

2-(4-Oxo-2-thioxo-thiazolidin-5-ylidenemethyl)-7,8-dihydro-6H-5-oxa-9-aza-benzocycloheptene-9-carboxylic acid p-tolylamide. MS: M⁺−1=412 Da.

### Example 111

5-(9-Cyclopentanecarbonyl-6,7,8,9-tetrahydro-5-oxa-9-aza-benzocyclohepten-2-ylmethylene)-2-thioxo-thiazolidin-4-one. MS: M⁺−1=375 Da.

### Example 112

5-[9-(2-Thiophen-2-yl-acetyl)-6,7,8,9-tetrahydro-5-oxa-9-aza-benzocyclohepten-2-ylmethylene]-2-thioxo-thiazolidin-4-one. MS: M⁺−1=403 Da.

### Example 113

2-(4-Oxo-2-thioxo-thiazolidin-5-ylidenemethyl)-7,8-dihydro-6H-5-oxa-9-aza-benzocycloheptene-9-carboxylic acid (3,5-dimethoxy-phenyl)-amide. MS: M⁺−1=360 Da.

### Example 114

5-[9-(3,4-Dichloro-benzoyl)-6,7,8,9-tetrahydro-5-oxa-9-aza-benzocyclohepten-2-ylmethylene]-2-thioxo-thiazolidin-4-one. MS: M⁺−1=451 Da.

### Example 115

5-[9-(3-Phenyl-acryloyl)-6,7,8,9-tetrahydro-5-oxa-9-aza-benzocyclohepten-2-ylmethylene]-2-thioxo-thiazolidin-4-one. MS: M⁺−1=409 Da.

### Example 116

5-[9-(Benzofurazan-5-carbonyl)-6,7,8,9-tetrahydro-5-oxa-9-aza-benzocyclohepten-2-ylmethylene]-2-thioxo-thiazolidin-4-one. MS: M-1=438 Da.

### Example 117

5-[9-(Pyridine-3-carbonyl)-6,7,8,9-tetrahydro-5-oxa-9-aza-benzocyclohepten-2-ylmethylene]-2-thioxo-thiazolidin-4-one. MS: M⁺−1=397 Da.

### Example 118

2-(4-Oxo-2-thioxo-thiazolidin-5-ylidenemethyl)-7,8-dihydro-6H-5-oxa-9-aza-benzocycloheptene-9-carboxylic acid (4-phenoxy-phenyl)-amide. MS: M⁺−1=504 Da.

### Example 119

5-[9-(2-Benzyloxy-acetyl)-6,7,8,9-tetrahydro-5-oxa-9-aza-benzocyclohepten-2-ylmethylene]-2-thioxo-thiazolidin-4-one. MS: M⁺−1=427 Da.

### Example 120

2-(4-Oxo-2-thioxo-thiazolidin-5-ylidenemethyl)-7,8-dihydro-6H-5-oxa-9-aza-benzocycloheptene-9-carboxylic acid (4-chloro-2-trifluoromethyl-phenyl)-amide. MS: M⁺−1=514 Da.

### Example 121

4-[2-(4-Oxo-2-thioxo-thiazolidin-5-ylidenemethyl)-7,8-dihydro-6H-5-oxa-9-aza-benzocycloheptene-9-carbonyl]-benzonitrile. MS: M⁺−1=407 Da.

### Example 122

2-(4-Oxo-2-thioxo-thiazolidin-5-ylidenemethyl)-7,8-dihydro-6H-5-oxa-9-aza-benzocycloheptene-9-carboxylic acid (3-methoxy-phenyl)-amide. MS: M⁺−1=442 Da.

### Example 123

5-[9-(Furan-2-carbonyl)-6,7,8,9-tetrahydro-5-oxa-9-aza-benzocyclohepten-2-ylmethylene]-2-thioxo-thiazolidin-4-one. MS: M⁺−1=386 Da.

### Example 124

2-Thioxo-5-{9-[3-(3-trifluoromethyl-phenyl)-acryloyl]-6,7,8,9-tetrahydro-5-oxa-9-aza-benzocyclohepten-2-ylmethylene}-thiazolidin-4-one. MS: M⁺−1=477 Da.

### Example 125

5-[9-(5-tert-Butyl-2-methyl-furan-3-carbonyl)-6,7,8,9-tetrahydro-5-oxa-9-aza-benzocyclohepten-2-ylmethylene]-2-thioxo-thiazolidin-4-one. MS: M⁺−1=443 Da.

### Example 126

5-[9-(4-Hexyl-benzoyl)-6,7,8,9-tetrahydro-5-oxa-9-aza-benzocyclohepten-2-ylmethylene]-2-thioxo-thiazolidin-4-one. MS: M⁺−1=467 Da.

### Example 127

2-[2-(4-Oxo-2-thioxo-thiazolidin-5-ylidenemethyl)-7,8-dihydro-6H-5-oxa-9-aza-benzocycloheptene-9-carbonyl]-anthraquinone. MS: M⁺−1=513 Da.

### Biological Example 1

### PI3Kγ Protein Expression and Purification Protocol

Spodtera frugiperda cells, grown in ESF921 media, were coinfected with baculovirus expressing a glu-tagged p110 and baculovirus expressing an HA-tagged p110γ, at a 3:1 ratio of p110 baculovirus to p110γ baculovirus. Sf9 cells were grown to 1×10⁷ total cells/mL in 10L bioreactors and harvested 48-72 hours post infection. Samples of infected cells were then tested for expression of p101/p110γ PI3 kinase by immunoprecipitation and Western Blot analysis methods (see below).

To purify PI3Kγ, 4 volumes of room temperature hypotonic lysis buffer (1 mM MgCl₂, 1 mM DTT, 5 mM EGTA, 1 mM Pefabloc, 0.5 μM aprotinin, 1M leupeptin, 2 μM pepstatin, 5 μM E64, pH 8) per gram of cell paste, was poured onto frozen cell pellets with stirring, then lysed in a nitrogen “bomb” at 400 psi (599HC T316, Parr Instrument Co, Moline, Ill.). NaCl was added to 150 mM, and sodium cholate was added to 1% and mixed for another 45 minutes. The lysates were clarified by centrifugation for 25 minutes at 14,000 rpm. The lysates were then loaded over anti-glu-linked Protein-G Sepaharose beads (Covance Research Products, Richmond, Calif.) using 20 mL resin/50 g cell paste. The column was washed with 15 volumes of wash buffer (1 mM DTT, 0.2 mM EGTA, 1 mM Pefabloc, 0.5 μM aprotinin, 5 μM leupeptin, 2 μM pepstatin, 5 μM E64, 150 mM NaCl, 1% sodium cholate, pH 8). PI3Kγ was eluted with 6 column volumes of wash buffer that contain 100 μg/mL of a peptide that competes for binding of the glu tag. The column fractions with the eluted protein (determined by taking OD₂₈₀ readings) were collected and dialyzed in 0.2 mM EGTA, 1 mM DTT, 1 mM Pefabloc, 5 μM leupeptin, 0.5% sodium cholate, 150 mM NaCl, and 50% glycerol, pH 8. The fractions were stored at −80° C. until further use.

### Biological Example 2

### G Protein Subunits Expression

Spodtera frugiperda cells were coinfected with baculovirus expressing a glu-tagged G protein β₁ and baculovirus expressing a G protein β₂ at a 1:1 ratio of glu-tagged G protein β₁ baculovirus to G protein β₂ baculovirus. Sf9 cells are grown in 10 L bioreactors and harvested 48-72 hours post infection. Samples of infected cells were tested for G protein β₁/β₂ expression by Western Blot analysis, as described below. Cell lysates were homogenized and loaded onto a column of glu-tagged beads as in Biological Example 1 and competed off the column with a glu peptide as described in Biological Example 1.

### Biological Example 3

### Western Blot Analysis

Protein samples were run on an 8% Tris-Glycine gel and transferred to a 45 μM nitrocellulose membrane. The blots were then blocked with 5% bovine serum albumin (BSA) and 5% ovalbumin in TBST (50 mM Tris, 200 mM NaCl, 0.1% Tween 20, ph 7.4) for 1 hour at room temperature, and incubated overnight at 4° C. with primary antibody diluted 1:1000 in TBST with 0.5% BSA. The primary antibodies for the p110γ, p110α, p110β, p85α, G protein β₁, and G protein γ₂ subunits were purchased from Santa Cruz Biotechnology, Inc., Santa Cruz, Calif. The p101 subunit antibodies were developed at Research Genetics, Inc., Huntsville, Ala. based on a p101 peptide antigen.

After incubation with the primary antibody, the blots were washed in TBST and incubated for 2 hours at room temperaure with goat-anti-rabbit HRP conjugate (Bio-Rad Laboratories, Inc., Hercules, Calif., product Number 170-6515), diluted 1:10,000 in TBST with 0.5% BSA. The antibodies were detected with ECL™ detection reagents (Amersham Biosciences Corp., Piscataway, N.J.) and quantified on a Kodak ISO400F scanner.

### Biological Example 4

### Immunoprecipitation

100 μL of cell paste from Biological Example 1 or 2 was thawed and lysed on ice with 400 μL of hypotonic lysis buffer (25 mM tris, 1 mM DTT, 1 mM EDTA, 1 mM Pefabloc, 5 μM leupeptin, 5 μM E-64 (Roche), 1% Nonidet P40, pH 7.5-8). The lysate was incubated for 2 hours at room temperature with glu-tagged beads (Covance Research Products, Cambridge, England, product Number AFC-115P). The beads were washed 3 times in wash buffer (20 mM Tris, pH 7.8-8, 150 mM NaCl₂, 0.5% NP40) and the protein eluted off the beads by heating in 2 times sample buffer (Invitrogen Corporation, Carlsbad, Calif., product Number LC1676).

### Biological Example 5

### PI3Kγ In Vitro Kinase Assay

The inhibitory properties of the compounds in Table 1 were assayed in an in vitro PI3K assay. In a 96-well polypropylene plate, each well was spotted with 2 μL of 50 times the desired final concentration of compound in DMSO. Purified recombinant p101/p110γ protein (0.03 μg; ˜2.7 nM) and G protein β₁/γ₂ subunits (0.09 μg; −57.7 nM) for each reaction was combined in the assay buffer (30 mM HEPES, 100 mM NaCl, 1 mM EGTA, and 1 mM DTT). ATP and [γ-³²P-ATP] (0.09 μCi) were added to this mixture so that the final ATP concentration in the reaction was 20 μM. Lipid micelles were formed by sonicating phosphatidylinositol-4,5-diphosphate (PIP₂), phosphatidylethanolamine (PE), and Na-cholate in the assay buffer for 10 minutes, adding MgCL₂ and incubating on ice for 20 minutes, for final concentrations of 25 μM PIP₂, 300 μM PE, 0.02% Na-cholate, and 10 mM MgCl₂ in the reaction. The reactions were started by adding equal volumes lipid and enzyme mixture in a total volume of 50 μL, allowed to run for 20 minutes at room temperature, and stopped with 100 μL 75 mM H₃PO₄. The lipid product was transferred to a glass fiber filter plate and washed with 75 mM H₃PO₄ several times. The presence of radioactive lipid product (PIP₃) was measured by adding Wallac Optiphase mix to each well and counting in a Wallac 1450 Trilux plate reader (PerkinElmer Life Sciences Inc., Boston, Mass. 02118). The IC₅₀ for each compound tested is reported in μM in Table 1:

TABLE 1

| Ex No. | IC₅₀ (μM) |
|---|---|
| 1 | 0.056 |
| 2 | 0.0217 |
| 3 | 1.13 |
| 4 | 0.65 |
| 5 | 1.865 |
| 6 | 0.151 |
| 7 | 0.78 |
| 8 | 2.18 |
| 9 | 2.59 |
| 10 | 0.16 |
| 11 | 0.053 |
| 12 | 0.41 |
| 13 | 0.41 |
| 14 | 1.13 |
| 15 | 0.0073 |
| 16 | 0.08 |
| 17 | 0.003 |
| 18 | 0.004 |
| 19 | 0.055 |
| 20 | 0.073 |
| 21 | 0.1 |
| 22 | 4 |
| 23 | 0.101 |
| 24 | 0.56 |
| 25 | 0.245 |
| 26 | 0.545 |
| 27 | 2.02 |
| 28 | 0.385 |
| 29 | 0.57 |
| 30 | 0.735 |
| 31 | 0.009 |
| 32 | 4 |
| 33 | 0.014 |
| 34 | 0.004 |
| 35 | 0.036 |
| 36 | 0.005 |
| 37 | 0.007 |
| 38 | 0.025 |
| 39 | 0.014 |
| 40 | 0.012 |
| 41 | 0.065 |
| 42 | 0.15 |
| 43 | 0.565 |
| 44 | 0.018 |
| 45 | 0.76 |
| 46 | 0.372 |
| 47 | 0.044 |
| 48 | 0.715 |
| 49 | 0.245 |
| 50 | 0.007 |
| 51 | 0.86 |
| 52 | 0.115 |
| 53 | 0.265 |
| 54 | 0.445 |
| 55 | 0.005 |
| 56 | 0.225 |
| 57 | 0.095 |
| 58 | 0.032 |
| 59 | 0.125 |
| 60 | 0.33 |
| 61 | 0.56 |
| 62 | 4 |
| 63 | 0.145 |
| 64 | 4 |
| 65 | 0.017 |
| 66 | 0.08 |
| 67 | 0.039 |
| 68 | 0.008 |
| 69 | 0.006 |
| 70 | 0.031 |
| 71 | 0.935 |
| 72 | 0.174 |
| 73 | 0.115 |
| 74 | 0.115 |
| 75 | 0.014 |
| 76 | 0.033 |
| 77 | 0.002 |
| 78 | 0.005 |
| 79 | 0.009 |
| 80 | 0.002 |
| 81 | 3.759 |
| 82 | 0.001 |
| 83 | 0.027 |
| 84 | 0.084 |
| 85 | 0.012 |
| 86 | 0.179 |
| 87 | 0.12 |
| 88 | 0.465 |
| 89 | 0.200 |
| 90 | 1.034 |
| 91 | 0.090 |
| 92 | 0.033 |
| 93 | 0.012 |
| 94 | 0.010 |
| 95 | 0.008 |
| 96 | 0.005 |
| 97 | 1.610 |
| 98 | 0.025 |
| 99 | 0.275 |
| 100 | 0.535 |
| 101 | 0.02 |
| 102 | 0.037 |
| 103 | 0.445 |
| 104 | 0.115 |
| 105 | 0.424 |
| 106 | 0.400 |
| 107 | 0.704 |
| 108 | 0.640 |
| 109 | 2.035 |
| 110 | 3.495 |
| 111 | 1 |
| 112 | 1.135 |
| 113 | 1.325 |
| 114 | 1.375 |
| 115 | 1.52 |
| 116 | 1.53 |
| 117 | 1.909 |
| 118 | 1.875 |
| 119 | 1.649 |
| 120 | 1.865 |
| 121 | 2.609 |
| 122 | 2.174 |
| 123 | 3.904 |
| 124 | 4.014 |
| 125 | 4.605 |
| 126 | 4.200 |
| 127 | 4.990 |

### Formulation Example 1

Tablet Formulation

| Ingredient | Amount |
|---|---|
| Compound of Formula I | 50 mg |
| Lactose | 80 mg |
| Cornstarch (for mix) | 10 mg |
| Cornstarch (for paste) | 8 mg |
| Magnesium Stearate (1%) | 2 mg |
| | 150 mg |

The compounds of the present invention (e.g., a compound of Formula I, or a pharmaceutically acceptable salt thereof) can be mixed with the lactose and cornstarch (for mix) and blended to uniformity to a powder. The cornstarch (for paste) is suspended in 6 mL of water and heated with stirring to form a paste. The paste is added to the mixed powder, and the mixture is granulated. The wet granules are passed through a No. 8 hard screen and dried at 50° C. The mixture is lubricated with 1% magnesium stearate and compressed into a tablet. The tablets are administered to a patient at the rate of 1 to 4 each day for treatment of a PI3K-mediated disorder or condition.

### Formulation Example 2

### Parenteral Solution

In a solution of 700 mL of propylene glycol and 200 mL of water for injection can be added 20.0 g of a compound of the present invention. The mixture is stirred, and the pH is adjusted to 5.5 with hydrochloric acid. The volume is adjusted to 1000 mL with water for injection. The solution is sterilized, filled into 5.0 mL ampules, each containing 2.0 mL (40 mg of invention compound), and sealed under nitrogen. The solution is administered by injection to a subject suffering from a PI3K-mediated disorder or condition and in need of treatment.

### Formulation Example 3

### Patch Formulation

Ten milligrams of a compound of the present invention can be mixed with 1 mL of propylene glycol and 2 mg of acrylic-based polymer adhesive containing a resinous cross-linking agent. The mixture is applied to an impermeable backing (30 cm²) and applied to the upper back of a patient for sustained release treatment of a PI3K-mediated disorder or condition.

It is understood that the examples and embodiments described herein are for illustrative purposes only and that various modifications or changes in light thereof will be suggested to persons skilled in the art and are to be included within the spirit and purview of this application and the scope of the appended claims. All publications, patents, and patent applications cited herein are hereby incorporated by reference in their entirety for all purposes.

What is claimed is:

1. A compound of Formula I: 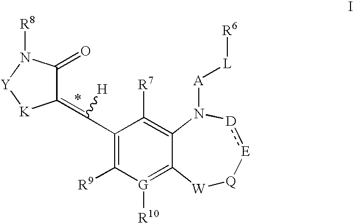
a pharmaceutically acceptable salt thereof;
or a pharmaceutically acceptable salt thereof;
wherein W is O, S, or NR²¹;
wherein R²¹ is selected from the group consisting of: —H, —CF₃, a C₁₋₆alkyl, and phenyl;
wherein Q is (CR²R³)ₚ,
wherein R² and R³ are independently selected from H or —CH₃;
wherein p is 0 or 1;
wherein E is CR⁴R⁵;
wherein R⁴ and R⁵ are independently selected from H or —CH₃;
wherein D is CR²⁸R³;
wherein R²⁸ and R³⁰ are independently selected from H or —CH₃;
wherein the dashed bond between D and E can be absent or present;
wherein A is absent, —S(O)₂—, —C(O)—, —C(O)—O—, —C(O)—NH—, or —C(S)—NH—;
wherein L is absent, a C₁-C₃-alkylene, —CH₂—, —(CH₂)₂—, —CH═CH—, a C₂-C₃-alkenylene, —CH₂—O—, —C₁-C₃-alkyl-O—, —CH₂—O—CH₂—, —C₁-C₃-alkyl-O—C₁-C₃-alkyl, —CH₂—S—, —C₁-C₃-alkyl-S—, C₁-C₃-alkyl-S(O)—, C₁-C₃-alkyl-S(O)₂—, —C₁-C₃-alkyl-S—C₁-C₃-alkyl-, —C₁-C₃alkyl-CO—,
C₁-C₃alkyl-C(O)O—, —C₁-C₃alkyl-C(O)—CH₂—, —C₁-C₃alkyl-C(O)NR²²—, —C₁-C₃alkyl-NR²²—C(O)—, —C₁-C₃alkyl-NR²²—C(O)—NR²⁴—, or —C₁-C₃alkyl-NR²²;
wherein R²² and R²⁴ are independently selected from H, and C₁₋₃alkyl;
wherein R⁶ is selected from the group consisting of H, a C₁₋₉alkyl, a C₂₋₉alkenyl, a C₂₋₉alkynyl, C(C₁-C₅alkyl)(C₁-C₅alkyl), a C₃-C₈cycloalkyl, a 3- to 8-membered heterocycloalkyl, a piperidinyl, a 6- to 12-membered bicyclic heterocycloalkyl, a 6- to 11-membered bridged bicyclic heterocycloalkyl, a 5-membered heteroaryl, a 5-isoxazolyl, a 3-isoxazolyl, an isoxazolyl, a 2-furanyl, a 3-furanyl, a 2-thienyl, a 3-thienyl, a thienyl, a 6-membered heteroaryl, a pyridinyl, a 4-pyridinyl, a 3-pyridinyl, an 8- to 12-membered bicyclic heteroaryl, a 2-quinoxalinyl, a quinoxalinyl, a phenyl, a naphthalenyl, a 1-naphthalenyl, a 2-naphthalenyl, a 9- to 12-membered bicyclic aryl, a 9,10-dioxo-9,10-dihydro-anthracen-2-yl, a benzofurazanyl, and a 4-(2,2-difluoro-1,3-benzodioxolyl;
wherein R⁷ is H, F, CF₃, or CH₃;
wherein R⁸ is H, —CH₂COOH, phenyl, —CH₃, a C₁₋₆alkyl, or a C₂₋₆alkenyl;
wherein Y is C(O), or C(S);
wherein K is NH, O, CH₂, or S;
wherein R⁹ is H, F, CF₃, or CH₃;
wherein G is C—R¹⁰ or N;
wherein R¹⁰ is H, —O—C₁₋₃alkyl, a C₁₋₃alkyl, —NO₂, —NR¹⁶R¹⁸, a —S—C₁₋₃alkyl, F or Cl;
wherein R¹⁶ and R¹⁸ are independently selected from the group consisting of: H, and C₁₋₃alkyl; and
wherein the stereochemistry of the double bond denoted “*” is entgegen or zusammen.

2. The compound of claim 1, wherein K is S, Y is C(S), and R⁸ is H.

3. The compound of claim 2, wherein W is 0, G is C—R¹⁰, p is 0, and R⁴, R⁵, R⁷, R⁸, R⁹, R¹⁰, R²⁸ and R³⁰ are H; and wherein the dashed bond between D and E is absent.

4. The compound of claim 1, wherein R⁶ is selected from the group consisting of H, a C₁₋₉alkyl, a C₂₋₉alkenyl, a C₂₋₉alkynyl, C(C₁-C₅alkyl)(C₁-C₅alkyl), a C₃-C₈cycloalkyl, a phenyl, a naphthalenyl, a 1-naphthalenyl, and a 2-naphthalenyl.

5. The compound of claim 1, wherein L is absent, a C₁-C₃-alkylene, —CH₂—, —(CH₂)₂—, —CH═CH—, a C₂-C₃-alkenylene, —CH₂—O—, —C₁-C₃-alkyl-O—, —CH₂—O—CH₂—, —C₁-C₃-alkyl-O—C₁-C₃-alkyl, —CH₂—S—, —C₁-C₃-alkyl-S—, or —C₁-C₃-alkyl-S—C₁-C₃-alkyl-.

6. The compound of claim 1, wherein A is —C(O)—, —C(O)—O—, or —C(O)—NH—.

7. The compound of claim 3, wherein R⁶ is H, a C₁₋₉alkyl, a C₂₋₉alkenyl, a C₂ galkynyl, C(C₁-C₅alkyl)(C₁-C₅alkyl), a C₃-C₈cycloalkyl, a phenyl, a naphthalenyl, a 1-naphthalenyl, or a 2-naphthalenyl.

8. The compound of claim 7, wherein L is absent, a C₁-C₃-alkylene, —CH₂—, —(CH₂)₂—, —CH═CH—, a C₂-C₃-alkenylene, —CH₂—O—, —C₁-C₃-alkyl-O—, —CH₂—O—CH₂—, —C₁-C₃-alkyl-O—C₁-C₃-alkyl-, —CH₂—S—, —C₁-C₃-alkyl-S—, or —C₁-C₃-alkyl-S—C₁-C₃-alkyl-.

9. The compound of claim 8, wherein R⁶ is H, a C₁₋₉alkyl, a C₂₋₉alkenyl, a C₂₋₉alkynyl, or a C(C₁-C₃alkyl)(C₁-C₅alkyl).

10. The compound of claim 9, wherein the compound is selected from the group consisting of:
5-(4-Isobutyryl-3,4-dihydro-2H-1,4-benzoxazin-6-ylmethylene)-2-thioxo-thiazolidin-4-one;
5-(4-Heptanoyl-3,4-dihydro-2H-1,4-benzoxazin-6-ylmethylene)-2-thioxo-thiazolidin-4-one;
8-Oxo-8-[6-(4-oxo-2-thioxo-thiazolidin-5-ylidenemethyl)-2,3-dihydro-1,4-benzoxazin-4-yl]-octanoic acid methyl ester; and
5-(4-Pentanoyl-3,4-dihydro-2H-1,4-benzoxazin-6-ylmethylene)-2-thioxo-thiazolidin-4-one.

11. The compound of claim 8, wherein R is a phenyl, a naphthalenyl, a 1-naphthalenyl, or a 2-naphthalenyl.

12. The compound of claim 9, wherein the compound is selected from the group consisting of:
4-[2-(3,4-Dichloro-phenyl)-acetyl]-3,4-dihydro-2H-benzo[1,4]oxazine-6-ylmethylene]-2-thioxo-thiazolidin-4-one;
6-(4-Oxo-2-thioxo-thiazolidin-5-ylidenemethyl)-2,3-dihydro-1,4-benzoxazine-4-carboxylic acid phenyl ester;
6-(4-Oxo-2-thioxo-thiazolidin-5-ylidenemethyl)-2,3-dihydro-1,4-benzoxazine-4-carboxylic acid p-tolyl ester;
5-[4-(3-Phenyl-acryloyl)-3,4-dihydro-2H-1,4-benzoxazin-6-ylmethylene]-2-thioxo-thiazolidin-4-one;
5-[4-(2-Benzyloxy-acetyl)-3,4-dihydro-2H-1,4-benzoxazin-6-ylmethylene]-2-thioxo-thiazolidin-4-one;
5-[4-(2-Phenylsulfanyl-acetyl)-3,4-dihydro-2H-1,4-benzoxazin-6-ylmethylene]-2-thioxo-thiazolidin-4-one;
6-(4-Oxo-2-thioxo-thiazolidin-5-ylidenemethyl)-2,3-dihydro-1,4-benzoxazine-4-carboxylic acid 4-methoxycarbonyl-phenyl ester;
6-(4-Oxo-2-thioxo-thiazolidin-5-ylidenemethyl)-2,3-dihydro-1,4-benzoxazine-4-carboxylic acid (3-trifluoromethyl-phenyl)-amide;
6-(4-Oxo-2-thioxo-thiazolidin-5-ylidenemethyl)-2,3-dihydro-1,4-benzoxazine-4-carboxylic acid phenethyl-amide;
6-(4-Oxo-2-thioxo-thiazolidin-5-ylidenemethyl)-2,3-dihydro-1,4-benzoxazine-4-carboxylic acid naphthalen-1-yl ester;
6-(4-Oxo-2-thioxo-thiazolidin-5-ylidenemethyl)-2,3-dihydro-benzo[1,4]oxazine-4-carboxylic acid (4-chloro-phenyl)-amide;
6-(4-Oxo-2-thioxo-thiazolidin-5-ylidenemethyl)-2,3-dihydro-benzo[1,4]oxazine-4-carboxylic acid (3,4-dichloro-phenyl)-amide;
6-(4-Oxo-2-thioxo-thiazolidin-5-ylidenemethyl)-2,3-dihydro-benzo[1,4]oxazine-4-carboxylic acid (3,5-dimethyl-phenyl)-amide; and
6-(4-Oxo-2-thioxo-thiazolidin-5-ylidenemethyl)-2,3-dihydro-benzo[1,4]oxazine-4-carboxylic acid (3-chloro-phenyl)-amide.

13. The compound of claim 8, wherein R⁶ is a C₃-C₈cycloalkyl.

14. The compound of claim 13, wherein the compound is selected from the group consisting of:
5-[4-(3-Cyclopentyl-propionyl)-3,4-dihydro-2H-1,4-benzoxazin-6-ylmethylene]-2-thioxo-thiazolidin-4-one;
6-(4-Oxo-2-thioxo-thiazolidin-5-ylidenemethyl)-2,3-dihydro-1,4-benzoxazine-4-carboxylic acid cyclopentylamide; and
5-[4-(3-Methyl-cyclohexanecarbonyl)-3,4-dihydro-2H-benzo[1,4]oxazin-6-ethylene]-2-thioxo-thiazolidin-4-one.

15. A method of treating a subject suffering from a PI3K-mediated disorder or condition comprising:
administering, to a subject suffering from a PI3K-mediated condition or disorder, a pharmaceutical composition comprising a therapeutically effective amount of a compound of claim 1 and a pharmaceutically acceptable carrier.

16. The method of claim 15, wherein said PI3K-mediated condition or disorder is selected from the group consisting of:
rheumatoid arthritis, osteoarthritis, inflammatory diseases, and autoimmune diseases.

17. The method of claim 15, wherein said PI3K-mediated condition or disorder is selected from the group consisting of:
cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease.

18. The method of claim 15, wherein said PI3K-mediated condition or disorder is selected from the group consisting of:
cancer, breast cancer, gliobastoma, endometrial carcinoma, heptocellular carcinoma, colon cancer, lung cancer, melanoma, renal cell carcinoma, thyroid carcinoma, small cell lung cancer, squamous cell lung carcinoma, glioma, breast cancer, prostate cancer, ovarian cancer, cervical cancer, leukemia, cell lymphoma, and lymphoproliferative disorders.

19. The method of claim 15, wherein said PI3K-mediated condition or disorder is selected from the group consisting of: type II diabetes.

20. The method of claim 15, wherein said PI3K-mediated condition or disorder is selected from the group consisting of:
respiratory diseases, bronchitis, asthma, and chronic obstructive pulmonary disease.

21. The method of claim 15, wherein said compound is a compound of any one of claims 1-14.

22. A pharmaceutical composition comprising:
a therapeutically effective amount of a compound of claim 1 and a pharmaceutically acceptable carrier.

23. A pharmaceutical composition comprising:
a therapeutically effective amount of a compound of claim 1-14 and a pharmaceutically acceptable carrier.
